SEQUENCE LISTING (1 sequence)

SEQ ID NO: 1 - RNA, 21 nt, Artificial Sequence
  Artificially synthesized miRNA
caacaccagucgaugggcugu

(19) United States
(12) Patent Application Publication
(10) Pub. No.: US 2022/0183973 A1
(43) Pub. Date: Jun. 16, 2022

(54) CAPTURE AND RELEASE GELS FOR OPTIMIZED STORAGE (CaRGOS) FOR BIOSPECIMENS

(71) Applicant: University of Louisville Research Foundation, Inc., Louisville, KY (US)

(72) Inventors: Gautam Gupta, Prospect, KY (US); Robert S. Keynton, Louisville, KY (US); Rajat Chauhan, Louisville, KY (US); Theodore Kalbfleisch, Louisville, KY (US)

(73) Assignee: University of Louisville Research Foundation, Inc., Louisville, KY (US)

(21) Appl. No.: 17/602,956

(22) PCT Filed: Apr. 13, 2020
(86) PCT No.: PCT/US2020/028009

§ 371 Date: Oct. 11, 2021

Related U.S. Application Data
(60) Provisional application No. 62/832,671, filed on Apr. 11, 2019.

(51) Int. Cl. A61K 9/127; A61K 47/02; A61K 47/10; A61K 47/18; A61K 9/06

(52) U.S. Cl. CPC A61K 9/127; A61K 47/02; A61K 9/06; A61K 47/183; A61K 47/10

(57) ABSTRACT

Provided are methods, compositions, and kits that are useful for long-term stabilization of biospecimens at ambient and elevated temperatures that are resilient to degradation by environmental factors and contaminants In some embodiments, the presently disclosed subject matter can be employed for long-term storage of biospecimens that would typically require low and/or ultra-low storage conditions, but as a consequence of employing the presently disclosed compositions and/or methods, the need for cryo-and/or sub-zero refrigeration is not needed in order to get similar if not superior stability of the biospecimen.

### CROSS REFERENCE TO RELATED APPLICATION

This application claims the benefit of U.S. Provisional Patent Application Ser. No. 62/832,671, filed Apr. 11, 2019, the disclosure of which is incorporated herein by reference in its entirety.

### TECHNICAL FIELD

The presently disclosed subject matter relates to a highly efficient sol-gel storage platform that allows for the long-term stabilization of biospecimens at refrigerated (4° C.), ambient, and elevated temperatures, with ˜100% single-step recovery. Also provided are methods, compositions, and kits useful for long-term stabilization of biospecimens at refrigerated, ambient, and elevated temperatures.

### BACKGROUND

Room temperature biospecimen storage in aqueous environment is a critical requirement in National Cancer Institute's (NCI) best practices for maximizing the quality of biospecimens against pre-analytical (collection, process and storage) degradation, prior to the downstream applications (Engel et al., 2014). A powerful yet conventional infrastructure of cryo-or refrigeration-based storage techniques is presently controlling the ‘biospecimen pre-analytical variables’ (BPV), in a pursuit of addressing ˜100% preservation of biospecimen-integrity and reproducibility in the downstream analyses (Engel et al., 2014). However, these conventional techniques require significant infrastructure and the expense associated with liquid nitrogen storage make it impractical for many laboratories and in field operations (Mutter et al., 2004; Fabre et al., 2013; Lou et al., 2014). Especially proteins and RNA samples are stored at sub-zero temperatures [−20, −80]° C. to avoid loss of total biospecimen integrity and unpredictability of biospecimen expression profiles during their downstream assays (Ibberson et al., 2009; Zhu et al., 2014; Harrill et al., 2016; Silsirivanit, 2019).

Furthermore, the studies generally indicate that sample degradation increases with storage time, and the freeze-thaw cycles negatively impact biospecimens as the formation of ice crystals results in physical shearing. Clearly, there is dearth of techniques that can store biospecimens at room temperature. Recent advances in room temperature storage include commercial products such as Biomatrica (San Diego, Calif., United States of America), GenTegra (IntegenX, Pleasanton, Calif., United States of America) and Imagene (Evry Cedex, France), which employ Anhydrobiosis (“life without water”); however, they are constrained with an extremely severe drying and rehydration stress, prior to the downstream processing (Kansagara et al., 2008; Martinez et al., 2010; Liu et al., 2015; Stevenson et al., 2015).

The potential for using hybrid functional materials to stabilize biological samples has been recognized by several scientists (Xiaolin et al., 2015). Encapsulation in silica sol gels, in particular, has been explored due to the relative simplicity and biocompatibility of the gels, and the ability to control its relative water content and salinity (Meng et al., 2010; Shen et al., 2011; Schlipf et al., 2013; Xiaolin et al., 2015; Chen et al., 2018). Despite interesting results over the last three decades, traditional sol-gel preparations are inherently complex, time-consuming, and require the use of acids or bases as a catalyst, along with alcohols as co-solvents; thus, they may be deleterious to biological samples, and therefore practical solutions compatible with current clinical practices have not been achieved. Another critical aspect of sol-gel immobilization is that the conventional techniques utilize extremely high concentration of silica precursors, that results in intact gels/glasses, however the recovery of biospecimen in solution remains extremely challenging and downstream processing is not feasible (Xiaolin et al., 2015). Although a higher degree of biospecimen-encapsulation is ubiquitous in higher concentration silica precursors, the biospecimen's integrity is gradually deteriorated among higher concentration sol-gel samples. Such deterioration or lack of ˜100% biospecimen recovery is attributed to the significant non-covalent interactions (e.g., electrostatic, van der walls, electronic) between sol-gel and biospecimen during their long-term storage (Vertegel et al., 2004; Shang et al., 2007; Meng et al., 2010; Shen et al., 2011; Schlipf et al., 2013; Cha et al., 2018; Chen et al., 2018; Peng et al., 2018).

An ideal host matrix for entrapping i.e. encapsulating and immobilizing biospecimen should therefore be (i) neutral aqueous solution with minimal chemical interactions with the biospecimen (ii) sterile and feasible to achieve with high reproducibility in any environment (iii) demonstrate intact biospecimen over long-term room temperature storage (iv) should possibly prevent the denaturation by proteases and nucleases that can arise from contamination (iv) easily amenable for biospecimen down-stream processing, and finally (vi) the process could be performed with minimal technical expertise. None of the current techniques can address all of these critical requirements.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for producing a Capture and Release Gel (CaRGOS) composition. In some embodiments, the methods comprise providing a solution of about 0.5 to about 20% (v/v) tetramethoxy silane (TMOS) and/or a derivative thereof, optionally wherein the solution is an aqueous solution of 0.5 to about 20% (v/v) tetramethoxy silane (TMOS) and/or a derivative thereof in water, optionally nuclease-free and/or protease-free water, or is a low salt aqueous solution, further optionally wherein the TMOS and/or the derivative thereof is at a concentration of about 0.5-10.0% (v/v); heating the solution for a time and at a temperature sufficient to solubilize and at least partially hydrolyze the TMOS and/or the derivative thereof in the solution, impart sterility to the solution, and/or evaporate all or substantially all methanol present and/or generated in the solution; and adding a buffer to the heated and at least partially hydrolyzed TMOS and/or derivative thereof, wherein the buffer comprises about 0.01-600 mM salt and/or has a pH of from about 5.0-9.0, and optionally further comprises 1-10 mM EDTA, to produce a buffered TMOS and/or derivative thereof solution, wherein a Capture and Release Gel (CaRGOS) composition is produced. In some embodiments, the heating step is performed in a microwave oven, optionally for about 15-120 seconds; and/or raises the temperature of the solution to in some embodiments at least about 40° C., in some embodiments at least about 42° C., in some embodiments at least about 45° C., in some embodiments at least about 50° C., in some embodiments at least about 55° C., in some embodiments at least about 60° C., in some embodiments at least about 64.5° C., in some embodiments at least about 70° C., in some embodiments at least about 75° C., in some embodiments at least about 80° C., in some embodiments at least about 85° C., in some embodiments at least about 90° C., in some embodiments at least about 95° C., or in some embodiments at least about 100° C.

In some embodiments, the CaRGOS composition further comprises a biospecimen. In some embodiments, the biospecimen is selected from the group consisting of a nucleic acid, optionally an RNA, further optionally a miRNA; a protein, optionally an antibody or a fragment or derivative thereof; a peptide, optionally a peptide hormone; a small molecule, optionally a small molecule drug; a liposome, optionally a liposome encapsulating an active agent; a forensic sample; and a cell and/or a lysate and/or a fraction thereof, or any combination thereof. In some embodiments, the pH of the CaRGOS composition is about 7.0-8.0, optionally about 7.4-7.6.

Also provided in some embodiments are CaRGOS compositions produced by the disclosed methods.

In some embodiments, the presently disclosed subject matter also relates to methods for stabilizing biospecimen again degradation. In some embodiments, the degradation is nuclease and/or protease degradation. In some embodiments, the methods comprise providing a buffered tetramethoxy silane (TMOS) and/or derivative solution, wherein the buffered (TMOS) and/or derivative solution is produced by providing a solution of about 0.5 to about 20% (v/v) tetramethoxy silane (TMOS) and/or a derivative thereof, optionally wherein the solution is an aqueous solution of 0.5 to about 20% (v/v) tetramethoxy silane (TMOS) and/or a derivative thereof in water, optionally nuclease-free and/or protease-free water, or is a low salt aqueous solution, further optionally wherein the TMOS and/or the derivative thereof is at a concentration of about 0.5-10.0% (v/v); heating the solution for a time and at a temperature sufficient to solubilize and at least partially hydrolyze the TMOS and/or the derivative thereof in the solution, impart sterility to the solution, and/or evaporate all or substantially all methanol present and/or generated in the solution; and adding a buffer to the heated and at least partially hydrolyzed TMOS and/or derivative thereof to produce a CaRGO composition, wherein the buffer comprises about 0.01-600 mM salt and/or has a pH of from about 5.0-9.0, and optionally further comprises 1-10 mM EDTA; and adding a biospecimen to the CaRGO composition, wherein the biospecimen is provided as an aqueous or low salt suspension or solution, whereby the biospecimen is stabilized against degradation. In some embodiments, the biospecimen is stabilized against nuclease and/or protease degradation. In some embodiments, the biospecimen is stabilized against degradation at a temperature of from about 4° C. to about 65° C. for at least 48 hours, for at least 1 week, for at least 2, weeks, or for at least 4 weeks relative to a biospecimen present in a solution that lacks the CaRGO composition.

In some embodiments, the presently disclosed subject matter relates to kits for storing degradation-sensitive biospecimens. In some embodiments, the kits comprise a first container comprising a solution of about 0.5 to about 20% (v/v) tetramethoxy silane (TMOS) and/or a derivative thereof, optionally wherein the solution is an aqueous solution of 0.5 to about 20% (v/v) tetramethoxy silane (TMOS) and/or a derivative thereof in water, optionally nuclease-free and/or protease-free water, or is a low salt aqueous solution, further optionally wherein the TMOS and/or the derivative thereof is at a concentration of about 0.5-10.0% (v/v); and optionally one or more of a low salt buffer comprising 0.05-0.6 M NaCl; and/or 1-1000 mM Tris-HCl (pH 5.0-9.0); and/or 1-10 mM EDTA; and/or nuclease-free and/or protease-free water, wherein the low salt buffer and the nuclease-free and/or protease-free water are present in separate containers; and instructions for using the contents of the kit for storing a nuclease-sensitive and/or protease-sensitive biospecimen.

The presently disclosed subject matter also relates in some embodiments to compositions for storing biospecimens. In some embodiments, the compositions comprise 0.5-20% (v/v) silicic acid; 0.05-0.6 M salt; and a buffer that maintains the composition at a pH of about 5.0-9.0. In some embodiments, the composition further comprises a biospecimen. In some embodiments, the biospecimen is selected from the group consisting of a nucleic acid, optionally an RNA, further optionally a miRNA; a protein, optionally an antibody or a fragment or derivative thereof a peptide, optionally a peptide hormone; a small molecule, optionally a small molecule drug; a liposome, optionally a liposome encapsulating an active agent; a forensic sample; and a cell and/or a lysate and/or a fraction thereof, or any combination thereof. In some embodiments, the biospecimen is a nucleic acid, and the silicic acid is present in the composition at a concentration of about 0.05-10% (v/v). In some embodiments, the biospecimen is a peptide or polypeptide, and the silicic acid is present in the composition at a concentration of about 5.0-20% (v/v). In some embodiments, the pH of the composition is lower than the pI of the peptide or polypeptide.

Thus, it is an object of the presently disclosed subject matter to provide methods and compositions for stabilizing biospecimens against nuclease and/or protease degradation.

An object of the presently disclosed subject matter having been stated hereinabove, and which is achieved in whole or in part by the presently disclosed subject matter, other objects will become evident as the description proceeds when taken in connection with the accompanying drawings as best described herein below.

### BRIEF DESCRIPTION OF THE FIGURES

FIGS. 1A-1C. Synthesis and Spectroscopic Characterization of CaRGOS. FIG. 1A is a schematic representation of an exemplary Sol-gel miRNA mixture preparation, incubation, separation, and characterization process. FIG. 1B is Raman spectra demonstrating complete TMOS hydrolysis within ˜30.0 seconds in conjunction with formation of methanol and silicic acid/dimers [Silicic acid: Si(OH)₄]. FIG. 1C is a graph showing ATR (Attenuated Total Reflectance) FT-IR spectroscopic analysis of CaRGOS aqueous formulations (0.5%, 0.8%, and 1.7% v/v) with an miRNA21 sequence (5′-CAACACCAGUCGAUGGGCUGU-3′; SEQ ID NO: 1).

FIGS. 2A-2C. Investigation of compatibility of CaRGOS with miRNA and hemoglobin. FIG. 2A is a bar graph of miRNA expression levels (CT) in CaRGOS (0.5% v/v) in low salt buffer and high salt buffer; CT 30 are equivalent to nuclease-free water. FIG. 2B is a representative schematic of the significant electrostatic-repulsions between negatively charged (-) silica-colloids and miRNA21. FIG. 2C is a plot of miRNA concentrations (nM) vs. CaRGOS percent concentrations (v/v) with their pH levels. Error bars in FIGS. 2A and 2C are±1 standard deviation from samples collected and analyzed in triplicate.

FIGS. 3A and 3B. Long-term evaluation of miRNA and hemoglobin expressions in CaRGOS. FIG. 3A is a plot of miRNA21 concentrations (nM) with sol-gel for 82 days at 4° C. (circles), 25° C. (squares), and 40° C. (triangles); miRNA21 concentrations (nM) without CaRGOS (Control) at 25° C. are also shown (inverted triangles). FIG. 3B is a graph of hemoglobin stabilities with incremental increase in CaRGOS concentrations (0-7.5% v/v). An unaltered UV-Vis absorbance band (406 nm) of heme group in hemoglobin framework was observed in CaRGOS formulations (5.0 and 7.5 v/v%). Error bars are ±1 standard deviation.

FIGS. 4A and 4B. Evaluation of stability in the presence of RNase. FIG. 4A is a schematic of the dual-character of negatively charged (−) silica-colloids demonstrating the electrostatic-attraction induced denaturation of positively charged RNase A and a simultaneous immobilization of miRNA21 within CaRGOS formulations via electrostatic repulsion. FIG. 4B is a plot of relative fluorescence intensity of Ethidium bromide against RNase A concentrations from 0-320 nM (squares).

FIGS. 5A and 5B. Polyethylene glycol (PEG) induced hemoglobin content release. FIG. 5A is a schematic of PEG addition to the CaRGOS formulation for facile hemoglobin extraction. FIG. 5B is a bar graph showing significant hemoglobin release in CaRGOS formulations (1.0-7.5% v/v) upon PEGylation. Error bars in FIG. 5B are ±1 standard deviation.

FIGS. 6A and 6B. Synthesis and Raman characterization of CaRGOS formulations. FIG. 6A is a schematic representation of CaRGOS formulations and encapsulation of hemoglobin for long-term room-temperature storage. FIG. 6B is a graph of complete hydrolysis of 5.0% v/v TMOS demonstrated by Raman spectra with an elimination of TMOS peak (646 cm⁻¹) and formation of methanol peak (1030 cm⁻¹) after a standard microwave synthesis.

### DETAILED DESCRIPTION

### I. General Considerations

Disclosed herein are simple room temperature storage technologies using capture and release gels for optimized storage (CaRGOS) for biospecimens. The room temperature integrity preservation of the exemplary biomolecules miRNA21 and the metalloprotein hemoglobin, at ambient as well as physiological temperatures under aqueous conditions, similar to their biological environment, are disclosed. The miRNA21 is a potential biomarker of tissue toxicity, cancer diagnosis, regulator of cancer immunotherapy biomarkers and down-regulator of multi-drug resistance (MDR) transporters (Harrill et al., 2016; Silsirivanit, 2019). Hemoglobin is a marker of oxidative injuries, anemia, hypertension, and renal toxicity, and is regularly used in clinics applications (e.g., blood donations, transfusions, etc.; Bursell & King, 2000). The sterile CaRGOS disclosed herein are achieved utilizing a deliberately ultra-low concentration of tetramethoxysilane/water suspension that is hydrolyzed in a standard microwave, typically for 30-60 seconds. Biospecimen (DNA, RNA, protein) of interest can be added to the hydrolyzed silica at room temperature, resulting in its stabilization.

Specifically, the room temperature integrity and preservation challenges using a representative highly sensitive bioanalytes miRNA21 and hemoglobin are disclosed herein. A single step ˜100% recovery of miRNA21 at room temperature using aqueous formulations of CaRGOS with extremely low silica concentrations (0.5%) has been demonstrated. The aqueous formulations of the CaRGOS with biospecimen are significantly versatile for downstream processing than conventional sol-gel matrices with immobilized biomolecular entities, that require physical or chemical methods to overcome the non-covalent interactions, with a strong likelihood of rupturing biological activity before downstream usage (Bursell & King, 2000; Kandimalla et al., 2006; Lee et al., 2012; Xiaolin et al., 2015). Although stabilization of biomolecular entities emanates from their restricted rotation or immobilization within the silica matrices, yet the highly aqueous formulations (0.5%) lack the concentration-range requisite for such immobilization. Therefore, to validate the mode of stabilization of biospecimen in highly aqueous formulations of CaRGOS matrices, the inherently dual nature of silica precursors-immobilization and nuclease-inhibition was tested. In the presently disclosed highly stable CaRGOS formulations, a remarkable resistance to nuclease (e.g., RNase A) was observed by demonstrating ˜0% quenching of ethidium bromide, thereby protecting ˜100% integrity of yeast RNA. Also, it is shown that a ˜69 nm hydrodynamic-sized aqueous formulation of CaRGOS (0.5%) efficiently preserved miRNA21 up to 82 days at above-freezing temperatures (e.g., 4° C., 25° C., and 40° C.) with ˜100% recovery in a single step. Moreover, the technique is completely compatible with a host of proteins as well as other nucleotides such as DNA.

Complementing the excellent preservation of a nucleic acid (miRNA21), the room-temperature stability and mechanical handling of a protein (hemoglobin) in nearly aqueous CaRGOS formulations (less than 5%) that preserved the protein's nativity, homogeneity, activity, and reproducibility over long-term storage is also disclosed herein. Using CaRGOS of 5% TMOS, greater than 95% of hemoglobin retained native structure for a period of 33 days at room temperature and up to 7 months at 4° C. Control groups (w/o CaRGOS) degraded significantly under similar conditions. The polyethylene glycol (PEG) release protocol allowed for 91% of the preserved-hemoglobin in 1% gels to be extracted via centrifuge.

Such strong stability of the hemoglobin content within CaRGOS formulations is strongly correlated to the scientific premise of isoelectric pH (pI) of proteins (Audain et al., 2016). A protein's solubility, stability, activity, and net charge [positive or negative] is heavily determined by the pI of the protein (Shaw et al., 2001). Hemoglobin, for instance, has pI of 6.8 and therefore incurs a negative charge at pH 8.2, and therefore it stayed soluble and is stable within the presently disclosed negatively charged silica formulations (pH 8.2). The hemoglobin stability in the presently disclosed CaRGOS formulations was attributed to two factors. Firstly, the electrostatic repulsions between negatively charged hemoglobin and silica might possibly be driving the protein stability within these colloidal dispersions. Secondly, the immobilization or restricted rotation of biospecimens imparted by silica formulations could stabilize the hemoglobin content. Immobilization, herein, is the result of either entrapping or collaterally depositing themselves alongside the native conformation of biospecimen (Chen et al., 2017). This immobilization is unique due to their conformation or shape recognizing capabilities, such that a congruent coupling of silica nanostructures occurs alongside the biospecimens (Chen et al., 2017). Therefore, the CaRGOS formulation techniques disclosed herein are applicable for preservation of most biomolecules, including but not limited to peptides, proteins, and nucleic acids.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Furthermore, the terms first, second, third, and the like as used herein are employed for distinguishing between similar elements and not necessarily for describing a sequential or chronological order. It is to be understood that the terms so used are interchangeable under appropriate circumstances and that the subject matter described herein is capable of operation in other sequences than described or illustrated herein.

Following long-standing patent law convention, the articles “a”, “an”, and “the” refer to “one or more” when used in this application, including in the claims. For example, the phrase “a cell” refers to one or more cells. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

### III. Compositions

In some embodiments, the presently disclosed subject matter relates to compositions that can be employed for stabilizing biospecimens, including but not limited to stabilizing the biospecimens for short- and/or long-term storage. As used herein, the term “stabilizing” and grammatic variants thereof refer to a state in which the biospecimen experiences less degradation (such as but not limited to degradation due to nuclease and/or protease activity on one or more components of the biospecimen) than would have occurred had the biospecimen not been stored in the composition of the presently disclosed subject matter. With respect to the stability from degradation, in some embodiments the specimen comprises, consists essentially of, or consists of a nucleic acid, in which case the relevant degradation is degradation resulting from nuclease activity. In some embodiments, the specimen comprises, consists essentially of, or consists of a peptide or polypeptide, in which case the relevant degradation is degradation resulting from protease activity. It is noted, however, that degradation of nucleic acids and peptides/polypeptides can also occur based on the presence of other activities that are not nuclease-based or protease-based but that results in damage to a nucleotide and/or phosphodiester backbone thereof and/or an amino acid and/or a peptide bond thereof. As such, the compositions of the presently disclosed subject matter are understood to stabilize biospecimens during short- and/or long-term storage against any form of degradation.

By way of example and not limitation, the stabilization provided results in no more than 10%, 9%, 8%, 7%, 6%, 5%, 4%, 3%, 2%, 1%, 0.5%, 0.25%, 0.1%, or 0.05% degradation of any type of the biospecimen over short- or long-term storage. The short or long term storage can be for a matter of days (e.g., 1, 2, 3, 4, 5, 6, 7, 8, 9, 10, 15, 20, 25, or 30 days), for a matter of weeks (e.g., 1, 2, 3, 4, 5, 6, 7, 8, 9, or 10 weeks), for a matter of months (e.g., 1, 2,3, 4, 5, 6, 7, 8, 9, 10, 11, or 12 months), or for a matter of years (e.g., 1, 2, 3, 4, or 5 years), or for longer. Additionally, the temperatures at which the short- or long-term storage can occur can be any temperature from about −20° C. to 4° C., up to and including room temperature (e.g., about 25° C.), to higher temperatures including 30° C., 35° C., 40° C., 45° C., 50° C., 55° C., 60° C., 65° C., or even greater than 65° C.

As used herein, the term “biospecimen” refers to any biomolecule or plurality of biomolecules for which the compositions and methods of the presently disclosed subject matter might be applicable. By way of example and not limitation, the term “biospecimen” includes nucleotides such as but not limited to RNA and DNA, proteins such as but not limited to enzymes, hemoglobin, and antibodies (including polyclonal and monoclonal antibodies, fragments thereof, and derivatives thereof); small molecule drugs, forensic samples, cells, including both eukaryotic and prokaryotic cells as well as lysates and fractions thereof, etc. In some embodiments, a biospecimen is a peptide hormone. In some embodiments, a biospecimen is a liposome, which in some embodiments can be a liposome encapsulating an active agent. As used herein, the term “active agent” refers to any bioactive molecule for which delivery to a subject, such as but not limited to delivery via a liposome, might be desired. Exemplary active agents include therapeutic agents, diagnostic agents, and detectable agents.

As used herein, the phrase “long-term stabilization” and grammatical variants thereof refers to storage conditions of temperature and duration that exceed in some embodiments, 2 days, in some embodiments 3 days, in some embodiments 5 days, in some embodiments 7 days, in some embodiments 14 days, in some embodiments 21 days, in some embodiments one month, in some embodiments two months, in some embodiments three months, in some embodiments six months, in some embodiments nine months, in some embodiments one year, and in some embodiments longer than one year.

Using the compositions and methods disclosed herein, it is possible to provide to a stored material (such as but not limited to a biospecimen) with greater stability than that stored material would have had under similar conditions of temperature and duration but in the absence of the use of the presently disclosed composition and methods. As used herein, the phrase “greater stability” refers to a degree of degradation of a biospecimen that is less than that which would have occurred had the biospecimen not been treated with the compositions and/or methods of the presently disclosed subject matter. By way of example and not limitation, the degree of degradation of the biospecimen treated with the compositions and/or methods of the presently disclosed subject matter is in some embodiments less than 95%, in some embodiments less than 90%, in some embodiments less than 85%, in some embodiments less than 80%, in some embodiments less than 75%, in some embodiments less than 70%, in some embodiments less than 65%, in some embodiments less than 60%, in some embodiments less than 55%, in some embodiments less than 50%, in some embodiments less than 45%, in some embodiments less than 40%, in some embodiments less than 35%, in some embodiments less than 30%, in some embodiments less than 25%, in some embodiments less than 20%, in some embodiments less than 15%, in some embodiments less than 10%, in some embodiments less than 5%, in some embodiments less than 4%, in some embodiments less than 3%, in some embodiments less than 2%, in some embodiments less than 1%, in some embodiments less than 0.5%, in some embodiments less than 0.1%, and in some embodiments less than 0.05%, of that which would have occurred had the biospecimen not been treated with the compositions and/or methods of the presently disclosed subject matter. In some embodiments, the degradation of the biospecimen that occurs and/or that would have occurred results or would have resulted from the presence of a contaminant, optionally a nuclease protease, and/or other enzyme. By way of example and not limitation, in some embodiments a contaminant is a nuclease, such as but not limited to a deoxyribonuclease and/or a ribonuclease (including but not limited to an RNase A), or a protease.

As used herein the phrase “TMOS and/or a derivative thereof” and grammatical variants thereof refers to tetramethyl orthosilicate (TMOS) and/or a derivative of TMOS. By way of example and not limitation, commonly known derivatives of TMOS are obtained by changing methyl group in TMOS to alkyl groups and/or chelating agents. Examples of such derivatives include but are not limited to where the alkyl groups (e.g., ethyl, propyl, butyl, pentyl, and hexyl) and chelating agents (e.g., EDTA). By way of particular example and not limitation, in some embodiments a chelating agent derivative of TMOS is TMS-EDTA (i.e., N-(trimethoxysilylpropyl)ethylenediamine triacetic acid trisodium salt). In some embodiments, the compositions that act as stabilizers are referred to herein as Capture and Release Gels for Optimized Storage (CaRGOS). CaRGOS are sol-gels that are formed from TMOS and/or derivatives thereof by hydrolysis followed by condensation as described herein. Biospecimens can be added to CaRGOS in order to stabilize the biospecimens from degradation during short or long term storage at various temperatures. In some embodiments, the temperature employed is a temperature that, in the absence of the CaRGOS, the biospecimen would be expected to suffer at least some degradation.

As such, in some embodiments the presently disclosed subject matter relates to CaRGOS compositions, including but not limited to those produced by the methods disclosed herein.

By way of example and not limitation, a CaRGO composition of the presently disclosed subject matter comprises 0.5-40% (v/v) silicic acid and/or a derivative thereof, which in some embodiments is produced by partially or completely hydrolyzing tetramethyl orthosilicate (TMOS) and/or a derivative thereof. Exemplary derivatives of TMOS include trimethoxy methyl silane, trimethoxy octyl silane, trimethoxy amino silane, and trimethoxy carboxylic silane.

Depending on the biospecimen for which the CaRGO composition of the presently disclosed subject matter is to be employed, the concentration of the silicic acid and/or the derivative thereof can be adjusted as desired. In embodiments in which the biospecimen is a nucleic acid, the CaRGO composition can comprises in some embodiments 0.5-40% (v/v) silicic acid and/or a derivative thereof, in some embodiments 0.5-20% (v/v) silicic acid and/or a derivative thereof, in some embodiments 0.5-15% (v/v) silicic acid and/or a derivative thereof, in some embodiments 0.5-10% (v/v) silicic acid and/or a derivative thereof, in some embodiments 1.0-10% (v/v) silicic acid and/or a derivative thereof, in some embodiments 1.0-5% (v/v) silicic acid and/or a derivative thereof, in some embodiments 1.5-10% (v/v) silicic acid and/or a derivative thereof, and in some embodiments 1.5-5.0% (v/v) silicic acid and/or a derivative thereof. In embodiments in which the biospecimen is a peptide or polypeptide, the CaRGO composition can comprises in some embodiments 0.5-40% (v/v) silicic acid and/or a derivative thereof, in some embodiments 0.5-20% (v/v) silicic acid and/or a derivative thereof, in some embodiments 0.5-15% (v/v) silicic acid and/or a derivative thereof, in some embodiments 0.5-10% (v/v) silicic acid and/or a derivative thereof, in some embodiments 1.0-10% (v/v) silicic acid and/or a derivative thereof, in some embodiments 1.0-5% (v/v) silicic acid and/or a derivative thereof, in some embodiments 1.5-10% (v/v) silicic acid and/or a derivative thereof, in some embodiments 1.5-5.0% (v/v) silicic acid and/or a derivative thereof, in some embodiments 5.0-10.0% (v/v) silicic acid and/or a derivative thereof, in some embodiments 5.0-15.0% (v/v) silicic acid and/or a derivative thereof, in some embodiments 5.0-20.0% (v/v) silicic acid and/or a derivative thereof, and in some embodiments 20.0-40.0% (v/v) silicic acid and/or a derivative thereof.

A CaRGO composition in some embodiments comprises a low salt concentration. As used herein, the phrase “low salt” refers to a salt concentration, in some embodiments a monovalent cation concentration, that is in some embodiments less than about 0.6 M, in some embodiments less than about 0.5 M, in some embodiments less than about 0.4 M, in some embodiments less than about 0.3 M, in some embodiments less than about 0.25 M, in some embodiments less than about 0.2 M, in some embodiments less than about 015 M, in some embodiments less than about 0.1 M, in some embodiments less than about 0.05 M, in some embodiments less than about 0.025 M, in some embodiments less than about 0.02 M, in some embodiments less than about 0.015 M, in some embodiments less than about 0.01 M, in some embodiments less than about 0.005 M, and in some embodiments about 0.00 M. In some embodiments, the salt is sodium chloride (NaCl), but other salts can also be employed in the CaRGO compositions disclosed herein.

A CaRGO composition also comprises a buffer. Any buffer that provide adequate buffering capacity in the pH range of about 5.0 to about 9.0 can be employed in the CaRGOS of the presently disclosed subject matter. An exemplary, non-limiting buffer system is based on 2-Amino-2-(hydroxymethyl)-1,3-propanediol (CAS Number 77-86-1; also referred to as THAM, Tris base, and Tris(hydroxymethyl)aminomethane), which is sold by various commercial suppliers under the trade name TRIZMA® base. Tris base can be pH adjusted using, for example hydrochloric acid to produce Tris-HCl at various pHs, or can be purchased as a pH-adjusted solution of various concentrations. Irrespective of the buffer system chosen, in some embodiments the CaRGO composition both before adding a biospecimen and after is characterized by a pH of about 5.0 to about 9.0, which depending on the desired use, can also have a near physiological pH. As used herein, the term “near physiological pH refers to a pH that is in some embodiments about 7.0, in some embodiments about 7.1, in some embodiments about 7.2, in some embodiments about 7.3, in some embodiments about 7.4, in some embodiments about 7.5, in some embodiments about 7.6, in some embodiments about 7.7, in some embodiments about 7.8, in some embodiments about 7.9, and in some embodiments about 8.0, or any pH value between about 7.0 and about 8.0. Any other buffer system that can provide a pH in the range of about 5.0 to about 9.0 can also be employed.

In some embodiments, the CaRGOS compositions of the presently disclosed subject matter further comprise a biospecimen. In some embodiments, a biospecimen is added to the CaRGOS composition as a solution, which in some embodiments is an aqueous solution and/or a low salt solution. Any volume of a biospecimen solution can be added to a CaRGO solution of the presently disclosed subject matter, provided that after additional of the biospecimen, the biospecimen-containing CaRGO composition comprises about 0.5 to about 40% (w/v) silicic acid, 0.05-0.6 M salt, and has a pH of about 5.0-9.0. Additional discussion of making CaRGOS compositions of the presently disclosed subject matter are provided herein below.

### IV. Kits

In some embodiments, the presently disclosed subject matter relates to kits comprising the presently disclosed compositions and/or comprising reagents that can be employed in making and using the disclosed compositions.

In some embodiments, the kits of the presently disclosed subject matter include reagents that can be employed in the preparation of one or more CaRGOS. Thus, in some embodiments the kits of the presently disclosed subject matter comprise, consist essentially of, or consist of tetramethoxy silane (TMOS) and/or derivative composition. In some embodiments, the TMOS and/or derivative is present in the composition at a concentration of about 0.5 to about 10% (v/v) TMOS and/or the derivative thereof in an aqueous solution. In some embodiments, the aqueous solution is deionized water, optionally nuclease-free and/or protease-free water.

In order to provide the greatest flexibility with respect to the final concentration of the TMOS and/or the derivative thereof in the CaRGO to be produced, the concentration of the TMOS and/or the derivative thereof in an aqueous solution should be higher than the concentration desired in the CaRGO such that the TMOS and/or the derivative thereof in the aqueous solution can be diluted as desired, for example, with deionized water or another low salt aqueous solution.

In those embodiments in which the CaRGO to be produced will include other components, some or all of those other components can be included in a kit of the presently disclosed subject matter or can be provided from an external source. Exemplary additional components of a CaRGO include NaCl, EDTA, and low salt buffers such as but not limited to Tris-HCl. In some embodiments, solid sodium chloride is provided, and in some embodiments a concentrated stock of NaCl is provided. In some embodiments, the concentrated stock can comprise 0.05-0.6 M NaCl, with any concentration between these values inclusive being appropriate for the compositions and methods of the presently disclosed subject matter.

Similarly, in some embodiments the kit includes a buffer component, which in some embodiments can be a Tris-based buffer. In the kits of the presently disclosed subject matter, Tris base can be provided as a solid, or can be provided as a concentrated stock solution, which in some embodiments can be anywhere from 1-1000 mM Tris that has been adjusted to a near physiological pH. Exemplary near physiological pH values include anything from about 7.0 to about 8.0. Therefore, a stock solution can be a 1-1000 mM Tris-HCl solution that is in some embodiments pH 7.0, in some embodiments pH 7.1, in some embodiments pH 7.2, in some embodiments pH 7.3, in some embodiments pH 7.4, in some embodiments pH 7.5, in some embodiments pH 7.6, in some embodiments pH 7.7, in some embodiments pH 7.8, in some embodiments pH 7.9, and in some embodiments pH 8.0. It is understood that any pH value between 7.0 and 8.0 inclusive can be employed in the compositions and methods of the presently disclosed subject matter.

In some embodiments, the CaRGO to be prepared will comprise EDTA. EDTA can also be provided in the kit as a solid or, if desired, in an aqueous solution. Appropriate EDTA solutions include those with concentrations of from about 1 to about 10 mM EDTA, with all values between 1 and 10 mM inclusive being appropriate for the presently disclosed subject matter.

In some embodiments, the kits also provide water for diluting the reagents and/or preparing the compositions of the presently disclosed subject matter. In some embodiments, the water is nuclease-free and/or protease-free water.

In some embodiments, each component of the kits is present in a separate container. Thus, the TMOS and/or the derivative thereof, the NaCl and/or the concentrated solution thereof, the Tris base and/or the Tris-HCl solution thereof, and/or the EDTA and/or the concentrated solution there can be in separate containers in order to provide maximum flexibility with respect to the final concentrations of each of the components desired in the CaRGO to be produced. Alternatively or in addition, one or more of these components may be provided together in a premixed solution. An exemplary premixed solution can include, for example, 0.05-0.5 M NaCl, 1-1000 mM Tris-HCl (pH 7.0-8.0), and 1-10 mM EDTA, which can then be diluted as desired to produce the CaRGOS of the presently disclosed subject matter.

In some embodiments, the kits of the presently disclosed subject matter also provide instructions for using the contents of the kit for storing nuclease-sensitive and/or protease-sensitive biospecimens and/or directions for where to access this information (including, but not limited to a website address).

### V. Methods for Making and Methods for Using the Disclosed Compositions and Kits

In some embodiments, the presently disclosed subject matter relates to compositions that can be employed for stabilizing biospecimens for storage. Exemplary methdos for preparing the CaRGOS of the presently disclosed subject matter are provided in the EXAMPLE, and are summarized as follows.

Generally, a CaRGO of the presently disclosed subject matter is produced by first providing an aqueous or low salt solution of TMOS and/or a derivative thereof as disclosed herein. In some embodiments, the TMOS and/or the derivative thereof is present at a concentration of about 0.5 to about 20% (v/v) in aqueous solution, optionally wherein the solution is an aqueous solution of 0.5 to about 20% (v/v) tetramethoxy silane (TMOS) and/or a derivative thereof in water, optionally nuclease-free and/or protease-free water, or is a low salt aqueous solution. The TMOS and/or the derivative thereof can be present in the solution at a concentration of in some embodiments 0.5-20.0% (v/v), and in some embodiments is present in the solution at a concentration of about 0.5-10% (v/v). In some embodiments, the TMOS and/or the derivative thereof can be present in the solution at a concentration of greater than 20.0% (v/v), including but not limited to 21.0% (v/v), 22.0% (v/v), 23.0% (v/v), 24.0% (v/v), 25.0% (v/v), 26.0% (v/v), 2.7.0% (v/v), 28.0% (v/v), 29.0% (v/v), 30.0% (v/v), 31.0% (v/v), 32.0% (v/v), 33.0% (v/v), 34.0% (v/v), 35.0% (v/v), 36.0% (v/v), 3.7.0% (v/v), 38.0% (v/v), 39.0% (v/v), 40.0% (v/v), or greater than 40.0% (v/v), as well as all values between 20.0% (v/v) and 40.0% (v/v), inclusive.

After preparation of the aqueous or low salt solution of TMOS and/or the derivative thereof, the solution is heated for a time and at a temperature sufficient to solubilize and at least partially hydrolyze the TMOS and/or the derivative thereof in the solution and/or to impart sterility to the solution, and/or to remove some, all, or substantially all methanol present in and/or generated in the solution as a result of the hydrolysis of the TMOS and/or the derivative thereof, for example by evaporation. For example, the temperature sufficient to partially hydrolyze the TMOS and/or the derivative thereof is that temperature at which the methanolic byproduct that results from the hydrolysis boils, which in some embodiments is about 64.5° C. In some embodiments, complete hydrolysis is achieved by heating the solution to 100° C. (i.e., the boiling point of water).

The time sufficient to solubilize and completely hydrolyze the TMOS and/or the derivative thereof is in some embodiments at least about 10, 15, 20, 25, or 30 seconds at 100° C., although longer times can also be employed. Partial hydrolysis can occur if lower temperatures are employed (such as but not limited to less than 64.5° C.) and/or if the solution is kept at a particular temperature for less than 10, 15, 20, 25, or 30 seconds. For higher concentrations of TMOS and/or the derivative thereof, (including but not limited to greater than 10%, 15%, 20%, 25%, 30%, 35%, or 40% v/v) complete hydrolysis can be accomplished by extending the period at which the solution remains at elevated temperatures, including in some embodiments 30-60 seconds at greater than 64.5° C. (including, for example, 30-60 seconds at about 100° C.). Any method for heating the solution can be employed, including but not limited to microwaving the samples for approximately 10, 15, 20, 25, or 30 seconds or more.

Once the desired degree of hydrolysis is accomplished and some or all of the methanol produced is liberated from the solution, the heated and at least partially or completely hydrolyzed TMOS and/or derivative thereof is added to an aqueous and/or low salt buffer to produce a buffered TMOS and/or derivative solution. By way of example and not limitation, an aqueous and/or low salt buffer can be added to result in the following concentrations of salt and buffer: 0.01-0.60 M salt (including but not limited to NaCl), 1-1000 mM Tris-HCl, and if desired, 1-10 mM EDTA. The buffer added should render the buffered TMOS and/or derivative solution at a pH that is in some embodiments between 5.0 and 9.0, and in some embodiments between 7.0 and 8.0. Exemplary, non-limiting components of the buffered TMOS and/or derivative solution include about 0.15 M NaCl, about 10 mM Tris-HCl (pH 7.0-8.0), and about 1 mM EDTA, although other concnetrations and/or pHs of these components can be employed as well to create the buffered TMOS and/or derivative solution.

The buffered TMOS and/or derivative solution is then ready to accept a biospecimen. The biospecimen is in some embodiments provided as a suspension or a solution in water or a low salt buffer, and the solution chosen and the amount added are selected to render a biospecimen-containing CaRGO composition which in some embodiments has the following components: about 0.5 to about 20% (v/v) TMOS and/or a derivative thereof, optionally wherein the TMOS and/or the derivative thereof is at a concentration of about 0.5-10.0% (v/v); 0.05-0.6 M salt (optionally NaCl); and 1-1000 mM Tris-HCl pH 5.0-9.0 (optionally pH 7.0-8.0). If desired, a divalent cation chelator such as but not limited to EDTA can also be present, and if present, can be at a concentration of about 1-10 mM. Once the biospecimen suspension or solution is added to the buffered TMOS and/or derivative solution, a Capture and Release gel (CaRGOS) composition has been produced.

It is noted that in the preparation of the buffered TMOS and/or derivative solution and thereafter the CaRGO, the amounts of the low salt buffer that are added to the at least partially or completely hydrolyzed TMOS and/or derivative thereof and of the biospecimen suspension or solution added to the buffered TMOS and/or derivative solution are merely exemplary. Generally, any volumes of low salt buffer that are added to the at least partially or completely hydrolyzed TMOS and/or derivative thereof and of the biospecimen suspension or solution added to the buffered TMOS and/or derivative solution can be employed provided that the CaRGO produced has a final concentration of about 0.05-0.6 M salt, has a pH of about 5.0-9.0 (in some embodiments, a pH that is near physiological pH (e.g., from 7.0-8.0 inclusive)), and has a final concentration of TMOS and/or the derivative thereof of about 0.5 to about 20% v/v in order to provide stabilization of the biospecimen in the CaRGO.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the EXAMPLES

TAQMAN® MicroRNA Reverse Transcription Kit, Tris EDTA buffer, Bovine pancreatic RNase A, yeast RNA MW 5000-8000, ethidium bromide, sterile 15.0 ml centrifuge tubes, Tetramethyl orthosilicate (TMOS), sodium phosphate monobasic, sodium phosphate dibasic, UV-Vis cuvettes and Sodium chloride were purchased from Sigma Aldrich (Saint Louis, Missouri, United States of America). Nuclease free water was purchased from New England BioLabs (Ipswich, Mass., United States of America). miRNA21 (5′-CAACACCAGUCGAUGGGCUGU-3′; SEQ ID NO: 1) was purchased from IDT Technologies, Inc. (Coralville, Iowa, United States of America. qPCR tubes were purchased from USA Scientific, Inc. (Fresno, Calif., United States of America) and 96 well plates were purchased from Thermo Fisher Scientific Inc. (Waltham, Mass., United States of America). The Reverse Transcription thermal cycle was performed on an Eppendorf thermocycler (Eppendorf, Hauppauge, N.Y., United States of America). The Dynamic Light Scattering measurements (DLS) were acquired on a Zetasizer (Zetasizer Nano ZS90, Malvern Instruments Ltd., Westborough, Mass., United States of America). The Zeta potential measurements were acquired on latter samples using a NanoBrook Zeta PALS Zeta Potential Analyzer (Brookhaven Instruments, Holtsville, N.Y., United States of America). Fluorescence measurements were acquired on Molecular Devices SpectraMax M2 plate reader (San Jose, Calif., United States of America) and modulus fluorimeter's green module with emission range: 580-640 nm (Sunnyvale, Calif., United States of America). FT-IR spectra were measured with the FT-IR spectrometer (PerkinElmer Spectrum 100, PerkinElmer, Inc., Waltham, Mass., United States of America) with universal ATR (attenuated total reflectance) sample accessory. Raman spectra were acquired on Reva Educational Raman platform (Hellma USA Inc., Plainview, N.Y., United States of America).

Degradation of Yeast RNA with enhancement of RNase A concentration. The degradation of yeast RNA with respect to change in the concentration of bovine pancreatic RNase A was monitored at pH 7.5 (0.05 M Tris buffer) containing 0.1 M NaCl. The yeast RNA and EtBr solutions were mixed and incubated for 30 min. A 2.7 ml of pH 7.46 “CaRGOS Buffer” [1:1:1 volume ratio of (A) CaRGOS (1.5% v/v; (B) 0.05 M Tris buffer with 0.1 M NaCl/pH 7.5 and (C) Nuclease free water; pH 7.46] or “Control Buffer” [0.05 M Tris buffer with 0.1 M NaCl; pH 7.5] were mixed with 0.2 ml (1 mg/ml RNA with 0.077 mM EtBr) and incubated for 100 s. These samples (with or w/o CaRGOS) were added into a respective well in a 96-well reaction plate and mixed gently to bring solution to the bottom of the wells. To the 96-well plates, 1-120 μl of 2.0 μM RNase A were added with the final volume to 200.0 μl and the change in fluorescence intensity monitored.

Reverse Transcription. Reverse Transcription (RT) master mix was prepared using the TAQMAN® MicroRNA Reverse Transcription Kit components before preparing the reaction. RT components were thawed on ice and 5X RT primers were vortexed. The 10 μL of Master mix-5X RT Primer was pipetted into a respective well in a 96-well reaction plate using 200 μL 96-well plate. The 5.0 μL of miRNA samples (with or w/o CaRGOS) were added into a respective well in a 96-well reaction plate, cap-sealed and mixed gently to bring solution to the bottom of the wells. The 96-well plates were further incubated on ice for 5 minutes and transferred to Eppendorf thermocycler at 85° C. for 65 minutes.

Real-Time qPCR Amplification. The 8.67 μL of master mix made for each miRNA21 (with or w/o CaRGOS) was pipetted into a 100 μL PCR 96-well reaction plate respective well. The 1.33 μL of RT product was transferred into respective 96-well reaction plate well, cap-sealed and gently mixed to bring solution to the bottom of the tube before real time qPCR amplification.

CaRGOS synthesis. 10.0% (v/v) TMOS stock-solution was prepared in de-ionized water and transferred to a 40.0 mL glass test tube, screw capped and hydrolyzed via microwave for thirty seconds. Post-microwave, the screwcap was removed to evaporate the volatile byproduct (i.e., methanol) of the CaRGOS synthesis. This CaRGOS stock solution was allowed to cool to room temperature. After room temperature was reached, appropriate amounts of CaRGOS were added to 4.0 mL cuvettes to create final concentrations (% (v/v)) of 0, 1, 2.5, 5 and 7.5 respectively. For miRNA encapsulation, Tris EDTA buffer [0.15 M NaCl, 10 mM Tris-HCl (pH 7.5), 1 mM EDTA, a payload of ˜500 nM miRNA21 concentration] was added. For encapsulation of hemoglobin, phosphate buffer (0.5 M, pH 8.2) was added to constitute the remainder of the 3 mL solution, as well as 0.03 mL of 1.0 w/v % hemoglobin.

Storage of Hemoglobin in CaRGOS. Several samples of CaRGOS-hemoglobin [(0.0-7.5)% (v/v) TMOS; 0.01 wt./v % Hemoglobin; 0.5 M Phosphate Buffer, pH 8.2; 3.0 mL] solutions were formulated in 4.0 mL UV-Vis cuvettes, capped and stored for a desired amount of time. The UV-Vis spectra of stored CaRGOS-hemoglobin [(0.0-7.5)% (v/v) TMOS; 0.01 wt./v % Hemoglobin; 0.5 M PB, pH 8.2; 3.0 mL] solutions were measured on 0, 2, 6, 9, 13, 18, 20, 24, 27, 31, 33 days at room-temperature, to validate integrity of hemoglobin in the CaRGOS. For long-term studies, we used optimized CaRGOS concentration [i.e., 5.0% (v/v) TMOS], while keeping rest of formulation parameter fixed [0.01 wt./v % Hemoglobin; 0.5 M PB, pH 8.2; 3.0 mL] and were measured over a period of 210 days, to validate integrity of hemoglobin over the prolonged room-temperature and refrigerated storage conditions.

Release of Hemoglobin from CaRGOS. Polyethylene glycol (65.0 μM, 1.0 mL) was added to 3 ml CaRGOS containing Hemoglobin for facile re-dissolution of the silica-dispersions. After vortexing the sample for 30 seconds 1.0 mL of the resulting solution was pipetted to a 15.0 mL centrifuge tube. This process was completed until a total of 5.0 mL PEG had been added to each sample, after which the remainder of the dissolved CaRGOS was pipetted into the 15.0 mL centrifuge tube. 3.0 mL of the dissolved CaRGOS was transferred to two 1.5 mL centrifuge tubes for each sample, after which they were centrifuged for 13 minutes at 10,000 rpm. The supernatant hemoglobin solution at the top of each tube was pipetted into the corresponding UV-Vis cuvette, where UV-Vis analysis was used to determine the concentration and structure of native hemoglobin.

Evaluation of CaRGOS evolution using Raman Spectroscopy. The Raman spectra was performed on CaRGOS [(0.0-10.0)% (v/v) TMOS], CaRGOS with buffer [0.5% (v/v) TMOS, 0.15 M NaCl, 10 mM Tris-HCl (pH 7.5), 1 mM EDTA], CaRGOS with buffer [(0.0-10.0)% (v/v) TMOS; 0.5 M PB, pH 8.2; 3.0 mL] and CaRGOS with hemoglobin/buffer [(0.0-7.5)% (v/v) TMOS; 0.01 wt./v % Hemoglobin; 0.5 M PB, pH 8.2; 3.0 mL] using a Reva Educational Raman platform (Hellma USA Inc., Plainview, N.Y., United States of America). The laser power of 450.0 mW and current 959.0 mA was optimized to analyze the samples. The laser temperatures [diode=30° C. ; Case=24.4° C.] and spectrometer temperature [23.1° C.] were optimized for collecting the Raman spectra.

### Example 1

### Synthesis and Spectroscopic Characterization of CaRGOS

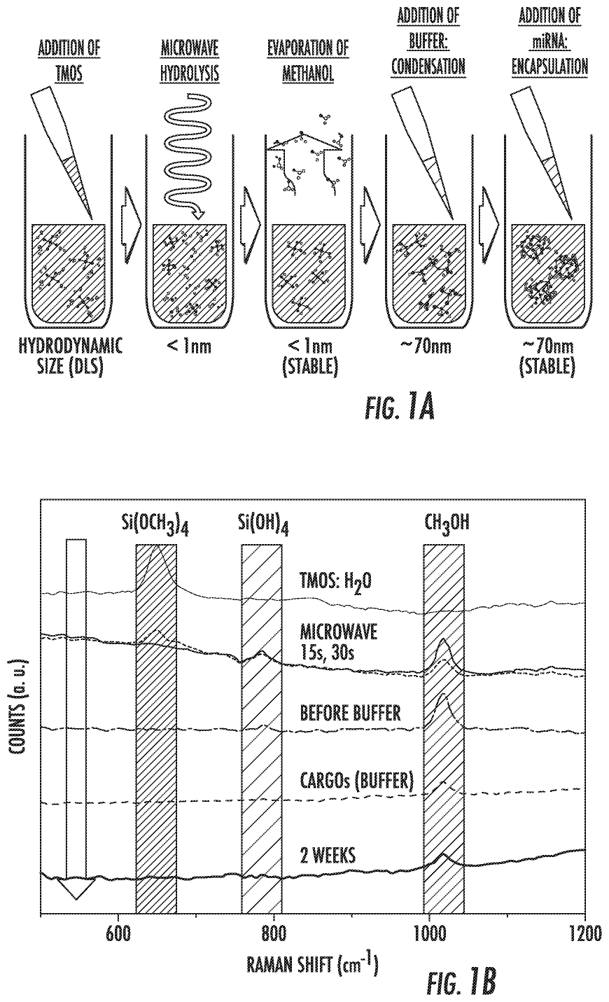

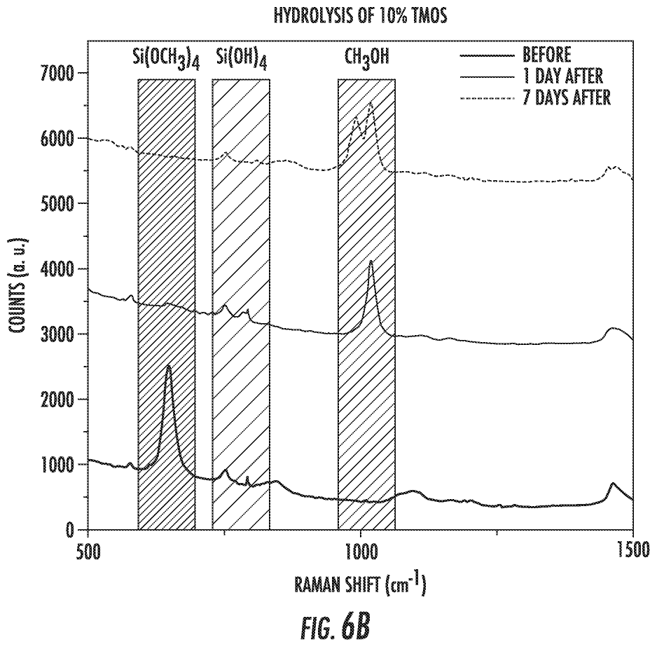

FIG. 1A shows step by step schematic of the formulation of CaRGOS developed for encapsulation of miRNA21 and FIG. 6A shows a schematic of CaRGOS process for encapsulation of hemoglobin. Typically, tetramethoxy silane in a desired concentration is mixed with deionized water. A standard microwave oven is used to impart mixing, induce hydrolysis (15-30 seconds) and simultaneous sterilization that results in the formation of Si(OH)₄ without the use of additional chemicals. The hydrolysis reaction generates undesired methanol byproduct that preferentially evaporates due to higher vapor pressure of methanol. However, a slight amount of methanol remains in the solution as confirmed by spectroscopic techniques but is not deleterious to the biospecimen. Biospecimens (miRNA, hemoglobin), buffer, and RNase-free water is further added and condensation reaction (formation of Si—O—Si) continues resulting in the stabilization of biospecimens. Specifically, the recovery of miRNA does not require any separation and is performed simply by taking an aliquot on a desired day, which is followed by RT-PCR studies to establish the quality and quantification of RNA. The process is extremely versatile, cheap, involves no use of acids and alcohols, and is amenable with just a standard microwave and requires minimum expertise.

Raman and IR Spectroscopy along with Dynamic light scattering (DLS) were utilized to understand the evolution of hydrolysis and condensation reactions in the silica precursor solutions. FIG. 1B and FIG. 6B show step by step Raman spectra of the aqueous formulations over a period of time. The theoretical peak positions of TMOS precursor (Si(OCH₃)₄), intermediates [(Si(OCH₃)₃OH, Si(OCH₃)₂(OH)₂), Si(OCH₃)(OH)₃)], silicic acid (Si(OH)₄, and methanol (CH₃OH) are expected at 640-650 cm⁻¹, 673-725 cm⁻¹, 750-780 cm⁻¹, and 1020 cm⁻¹, respectively (Zerda & Hoang, 1989). Experimentally, a peak was observed at 646 cm⁻¹ for 1.25% TMOS/water solution prior to microwave irradiation exposure. After 15 seconds of exposure, this peak gradually decreased, and intermediate/methanol peaks were observed at 750-780 cm⁻¹ and 1020 cm⁻¹. After 30 seconds of exposure to microwaves, the TMOS peak completely disappeared indicating complete hydrolysis, and an increase in Si(OH)₄/dimer and methanol peaks at 780 cm⁻¹ and 1020 cm⁻¹ were observed. The efficiency of the hydrolysis was computed utilizing the Raman peak of methanol aqueous solutions. The hydrolyzed precursor exhibited sufficient stability and was utilized to stabilize any biospecimen of choice. Buffer was subsequently added to CaRGOS solution, and a decrease in methanol peak was observed due to subsequent dilution. In addition, the peak at 780 cm⁻¹ completely disappeared indicating a change in the structure of the Si(OH)₄/dimer. In the final solution, the methanol concentration was estimated to be around 80 μM.

Raman spectra of TMOS (0.1-5.0% v/v) solutions, before and after the TMOS hydrolysis under microwave exposure, were also taken. Upon addition of the buffer containing hemoglobin, the intensity of methanol peak decreased due to dilution and emergence of a peak at 980 cm⁻¹ was observed, which arose due to the presence of phosphate buffer.

The disappearance of silicic acid/dimer peak was carefully studied using Dynamic light scattering. Prior to the addition of buffer, around ˜1 nm hydrodynamic diameter was observed, indicating a possible absence of colloidal particles and therefore indicating presence of only silicic acid [Si(OH)₄] or some dimers in highly aqueous CaRGOS. Upon the addition of buffer, CaRGOS formulations displayed a transition from a ˜1 nm hydrodynamic-sized dispersion to highly monodisperse and ˜69 nm hydrodynamic-sized dispersion. Although not wishing to be bound by any particular theory of operation, this was possibly due to decrease in electrostatic repulsions between negatively charged silica precursors in presence of saline environment. The addition of biospecimen showed a negligible change in zeta potential, polydispersity index, and hydrodynamic diameter. Being viscous, DLS and zeta potential measurements were not possible for (1.5-5.0)% CaRGOS.

Since the concentrations of the silica precursor for biospecimen was extremely low, the IR signatures of low concentration gels were similar to pure water. Therefore, the concentration of silica precursor was gradually increased to observe the characteristics peaks of silica. FIG. 1C shows the IR spectra of miRNA-CaRGOS with variable silica concentration that indicate the increase in bands of 1085 cm⁻¹ (Si—O—Si asymmetric vibration) and 1045 cm⁻¹. (Si—OH asymmetric vibration) After complete spectroscopic investigation of hydrolysis and condensation of silica precursor, the compatibility of the presently disclosed CaRGOS process with miRNA21 and hemoglobin followed by long-term stability studies was investigated.

### Example 2

### Investigation of Compatibility of CaRGOS with miRNA and Hemoglobin

An ideal storage solution should have near physiological conditions and the key litmus test is to probe the compatibility of the CaRGOS with a sensitive biospecimen. For instance, the role of electrolytes in preservation of biospecimens is crucial, however usually understated (Pinto et al., 2014; Wagner-Golbs et al., 2019). Electrolyte composition (pH, ionic strength, concentration) can directly impact not only the biospecimen viability but also the silica stability over time. It should be noted that the ionic strength, pH of the CaRGOS, and the concentration of silica precursor can drastically impact the stability and release of biospecimen. For example, ionic strength of the solution can directly impact the electrostatic repulsions between negatively charged CaRGOS in buffer environment and consequently affects the size, stability, and monodispersity of the silica precursors. Also, salinity can dictate the nature of non-covalent interactions between biospecimen and CaRGOS matrices. Similarly, pH of the solution can dictate the extent of condensation reaction as well as the stability of biospecimen. Therefore, a systematic study was performed by varying these conditions and simultaneously monitoring the biospecimen expressions in each case respectively.

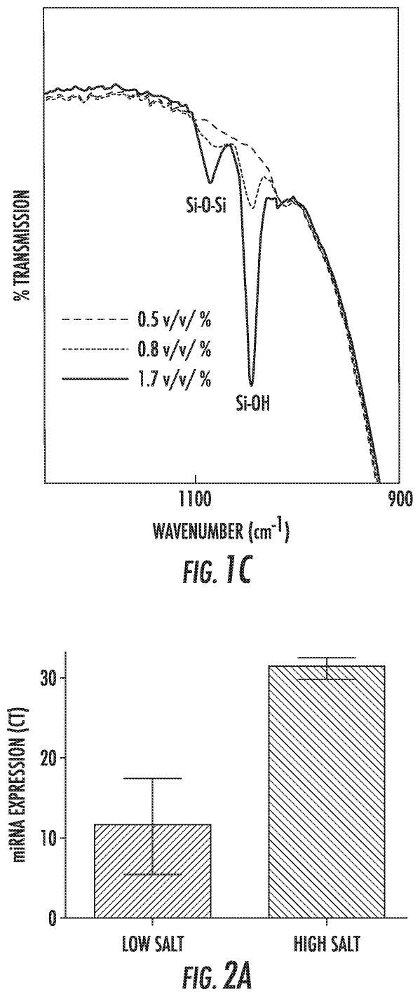

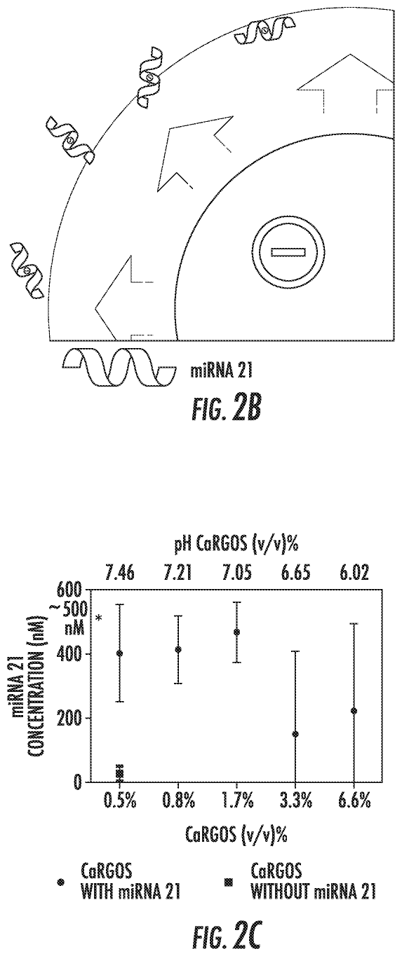

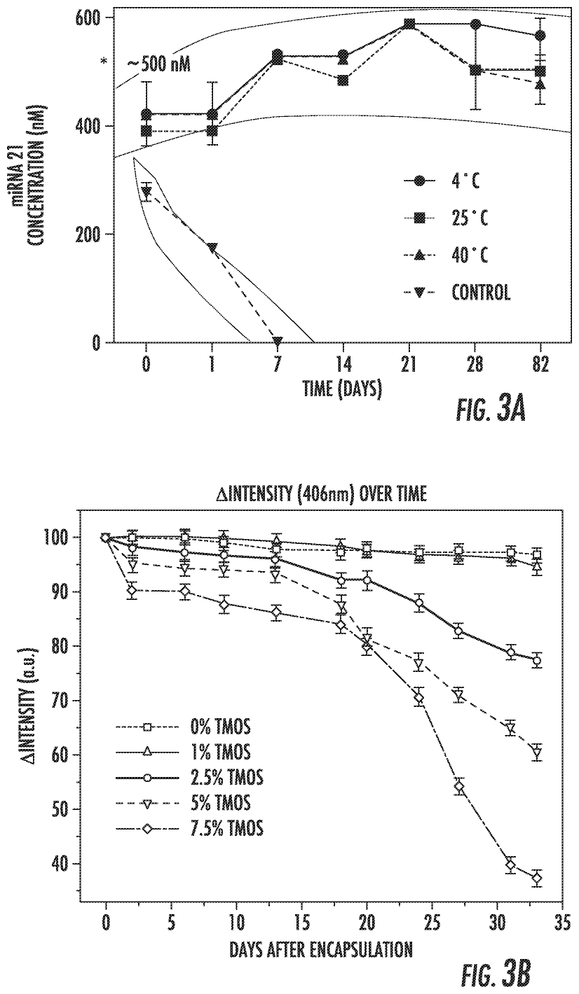

FIG. 2A shows the miRNA expression levels with 0.5% CaRGOS in Tris EDTA buffer with either 0.15 M NaCl or 0.5 M NaCl, while keeping fixed rest of the CaRGOS storage parameters [10 mM Tris-HCl (pH 7.5), 1 mM EDTA, a payload of ˜500 nM miRNA21 concentration]. Reverse transcription (RT) was performed on the miRNA21 sample aliquots (with and w/o CaRGOS) using TAQMAN® MicroRNA Reverse Transcription Kit and thermal cycler, followed by real-time quantitative polymerase chain reaction (qPCR) amplification with an applied 0.1 C_{T} threshold value. While the C_{T} values ≥30 were attributed to the nuclease-free water or small amount of nucleic acids generated by a sterile-compromised contaminated environment, the C_{T} values <30 were used to confirm miRNA expressions levels.

The miRNA21 concentration (nM) of CaRGOS aqueous formulations were evaluated by measuring mean C_{T} values using standard calibration curves. A strong correlation between miRNA21 concentration (nM) of CaRGOS aqueous formulations and mean C_{T} values was observed with regression equation C_{T}=−0.0437 [miRNA Conc(nM)]+28.926 and R²=0.99 respectively. In response to salt stress, the miRNA expression levels in low salt buffer were C_{T}: 11.5±6.0, whereas in high salt buffer the miRNA expressions were C_{T}: 31.1±1.2. These results indicated that the miRNA expression was compromised in high ionic strength buffer and the miRNA was completely viable in low salt buffer. Interestingly, these conditions are similar to biological pH and ionic strength with an extremely low concentration (0.5%) of silica. Under these conditions, the nucleotides have a negative charge and the silica colloids also exhibit a negative charge as well. Therefore, electrostatic repulsive forces dominate and the miRNA21 is stabilized. FIG. 2B shows a schematic of miRNA21 in CaRGOS. While not wishing to be bound by any particular theory of operation, it is possible that at such low concentrations of silica that were slightly viscous in nature, the miRNA21 could bounce slowly around silica colloids and would remain stable. These repulsive attractions also allow for ease of retrieval from CaRGOS without requirement of a separation step.

Once the concentration of salt was optimized, the concentration of silica precursor was varied. As the concentration of hydrolyzed silica (silicic acid) was increased, a slight decrease in pH was observed for the CaRGOS with buffer and biospecimen. A ˜500 nM payload of miRNA21 was added to each CaRGOS formulation. FIG. 2C shows the miRNA expression of miRNA (0-579 nM) on day 1 in CaRGOS prepared with variable TMOS concentrations. High miRNA expressions were observed only in lower silica concentrations [(0.5-1.7)% (v/v) (pH >7). However, at the higher concentrations of silica precursors, (pH<7) miRNA expression levels are slightly compromised. The drastically deviating miRNA21 expressions levels in ˜(0-527) nM range at higher CaRGOS concentrations [(3.0-7.0)% (v/v); pH<7] were tentatively attributed to the restricted mobility of miRNA in CaRGOS matrices due to the formation of highly viscous gels. High concentration CaRGOS matrices might restrict ease of miRNA retrieval for their downstream analyses. The augmented restricted mobility in CaRGOS sol-gel matrix was also manifested with the transition from weak to strong silica FT-IR bands in the increasingly viscous CaRGOS sol-gel formulations (Liu et al., 2015; FIG. 1C). Post-nucleotide analysis, the concentration gradient of CaRGOS (<10)% (v/v) formulations were tested for the preservation of a metalloprotein (hemoglobin). Hemoglobin binds and transport analytes (i.e., oxygen, nitric oxide, carbon monoxide) and plays significant role in the regulation of the blood pressure. Hemoglobin is a model protein of our choice for investigating the preservation of structural integrity under environmental stimuli (heat, mechanical excursions, nuclease/protease/microbial contamination), due to its complex four protein-chain framework, with each chain having heme group and metal center (i.e., iron) in the central cavity.

Purified proteins in their native state are known to be slightly disordered and for having certain sections in their unfolded state (Raynal et al., 2014). Therefore, instead of investigating secondary structures (i.e., α-helix), the thermal stability (˜25° C.) and mechanical handling (mixing, vortexing, shaking) investigations with CaRGOS-hemoglobin formulations were focused on the analysis of heme groups of the four-polypeptide chain network of hemoglobin. UV-Vis spectra can detect loss or alterations in heme and is an effective indicator of changes in primary and secondary structure (Zhu et al., 2002; Goodarzi et al., 2014). In addition, losses in heme and the resulting change in the secondary structure are indicative of alteration of tertiary structure conformation, as each of the subunits are integral to the tertiary structure of the molecule (Zhu et al., 2002). Therefore, the integrity of hemoglobin in CaRGOS was evaluated using UV-vis spectroscopy. UV-Vis spectroscopy is a rapid and routinely used method to validate structural stability of hemoglobin (λ=406 nm; heme group).

A sharp UV-Vis band (λ=406 nm) of the prosthetic heme group (C₃₄H₃₂O₄N₄Fe) in aqueous hemoglobin solutions (0.01 wt./v%; 0.5 M PB; pH 8.2) and in the CaRGOS (1.0-7.5)% (v/v) formulations immediately after immobilization was observed. This absorbance band indicated the excellent initial stability of hemoglobin in both the CaRGOS and control samples. The intact absorbance band observed also supported that silica condensation and the presence of the methanol by product did not affect hemoglobin nativity.

### Example 3

### Long-Term Evaluation of miRNA and Hemoglobin Expressions in CaRGOS

Given the significantly higher miRNA expressions levels in lower CaRGOS concentrations [(0.5-1.7)% (v/v); pH>7], these aqueous formulations of CaRGOS sol-gel matrix were utilized for long-term room/elevated temperatures miRNA21 storage. Three temperature conditions were utilized: (a) Refrigeration (4° C.); (b) ambient temperature (25° C.); and (c) near incubating cell-culture temperatures (40° C.), respectively. FIG. 3A shows the quantitative RT-PCR analysis of 0.5% (v/v) CaRGOS/miRNA21 mixtures that demonstrated ˜100% recovery of miRNA21 expression levels at 4° C., 25° C., and 40° C. over a period of 82 days. Though 1.7% (v/v) CaRGOS aqueous formulations had displayed relatively higher levels of miRNA21 expression levels in comparison to the 0.5% (v/v) CaRGOS, they were avoided for temperature dependent (4° C., 25° C., 40° C.) studies with qRT-PCR work flow, for being relatively viscous CaRGOS silica matrix thus requiring laborious pre-vortexing of CaRGOS/miRNA samples. At room temperature, the miRNA21 concentration (nM) in the mixtures without CaRGOS sol-gel matrices were (277.6±18.8) nM on day 0, (172.0±0.2) nM on day 1 and undetermined on day 7 (FIG. 3A). In contrast, nearly unaltered payload of miRNA21 concentrations (˜500 nM) were observed in the CaRGOS mixtures at room-temperature over 82-day period: (426.63±46.33) nM on day 0, (392.35±8.28) nM on day 1, (524.53±6.54) nM on day 7, (484.32±2.46) nM on day 14, (588.94±0.54) nM on day 21, (505.95±75.00) nM on day 28, and (500.19±59.92) nM on day 82 inferring a thermal stable miRNA21 within CaRGOS formulations. Low expression of miRNA on day 0 and day 1 was attributed to possible interference of free methanol during PCR amplification.

Similar results were observed for DNA preservation studies. The mode of miRNA stability in lower concentration CaRGOS sol-gel matrices might be attributed to the combined effect of some physical restriction of miRNA backbone mobility caused by sterically-restricted synergistic interactions with CaRGOS network and local solvent/pH microenvironment [Tris EDTA buffer/pH>7)] of these aqueous CaRGOS formulations (Carrasquilla et al., 2012; Perumal et al., 2014; Xiaolin et al., 2015). However, such long-term stability cannot simply arise from repulsive interaction, and other factors may play a critical role.

Tn intact absorbance band also indicated preservation of hemoglobin nativity in the CaRGOS (1.0-7.5)% (v/v) formulations immediately after immobilization. While maintaining constant hemoglobin concentration (0.01 wt./v %) and buffer environment (0.5 M PB, pH 8.2), the CaRGOS concentration (0.0-7.5)% (v/v) range were observed over a period of month. Relative to the control-group hemoglobin solutions (i.e., w/o CaRGOS), the data presented in FIG. 3B showed a two-fold [CaRGOS (1.0% (v/v))] and three-fold [CaRGOS (2.5% (v/v))] hemoglobin stability was observed in CaRGOS formulations. This demonstrated a CaRGOS-concentration dependent trend in determining the physical and chemical stability of hemoglobin.

FIG. 3B also shows that CaRGOS (5.0 and 7.5)% (v/v) solutions retained nearly ˜100% hemoglobin-stability up to 3-weeks and ˜95% stability for 33 days. Relatively high CaRGOS concentrations (5.0-7.5)% (v/v), were, therefore, ideal for storing hemoglobin under room-temperature and mechanical-handling (i.e., mixing, vortexing) based conditions. FIG. 3B also shows that control samples (i.e., w/o CaRGOS) at room-temperature had a significant decrease in UV-Vis absorbances: ˜10% in 1-week, ˜20% in 3-weeks and ˜63% in four weeks respectively.

Prolonged storage at refrigerated temperature and room-temperature of proteins is highly desirable for numerous medical applications. Prolonged storage (several months) studies were performed in a similar format to the 33-day hemoglobin storage described herein above. CaRGOS formulations (5.0 & 7.5% (v/v)) demonstrated exceptional hemoglobin storage capabilities over 1-month storage interval (FIG. 3B). However, the 5.0% (v/v) formulation was preferentially chosen over 7.5% (v/v) formulation towards investigating hemoglobin integrity over 210 days (7 month), attributing to an easier biospecimen passage/recovery through CaRGOS matrices, and less cost per sample. The optimized CaRGOS [(5.0% (v/v)) TMOS; 0.01 wt./v% Hemoglobin; 0.15 M PB, pH 8.2; 3.0 mL] solutions demonstrated an unprecedented hemoglobin-stability (˜96%) for at least a 7-month period at 4° C. under the non-sterile, room-temperature storage conditions. During prolonged refrigeration, the control group hemoglobin solutions (0.01% (wt/v); 0.15 M PB; pH 8.2) also displayed robust stability (˜96%) up to the 40-day period, demonstrating the short-term stabilizing effect of refrigeration as well as the phosphate buffer environment on control group hemoglobin solutions. However, escalated hemoglobin degradation over the long-term refrigeration period for control samples was observed, with a significant loss of heme group (406 nm) absorbance (˜70%). Under room temperature conditions, 5% CaRGOS samples retained 47% absorbance over the 210-day time period, while control samples retained 3% absorbance under the same conditions. This supported the long-term storage capabilities of 5% CaRGOS under both ambient room temperature and refrigerated conditions.

Analogous Raman spectra of the CaRGOS formulations (5.0% (v/v) TMOS; PB pH 8.0) with and w/o hemoglobin at days 7, 14, and 21 respectively. The Raman peaks of TMOS solution was assigned to 646 cm⁻¹, dimerized silica or silicic acid to 830 cm⁻¹, and the intense methanol C—O stretch to 1030 cm⁻¹, respectively. Similar peak intensities over 21 days were attributed to the robust physico-chemical stability of CaRGOS dispersions under room temperature and mechanical handling (i.e., mixing, shaking, vortexing) conditions. Also, the unaltered peak intensities of CaRGOS formulations, with and without hemoglobin, were attributed to the unique shape-recognition capabilities of silica nanostructures. Notably, the CaRGOS nanoformulations could potentially deposit around hemoglobin and match its shape/conformation, resulting in similar rotational and vibrational fingerprints of the CaRGOS formulations, with and without hemoglobin (Chen et al., 2017).

The human biological environment, for instance plasma, has unusually high concentration of proteins such as albumin, globulin, fibrinogen, and others (e.g., 60-80 mg/ml; Pinto et al., 2014; Wagner-Golbs et al., 2019). These 1,000,000 times increments in protein concentration as compared concentrations of hemoglobin (˜nanomolars) had presented a significant risk to the clinical translation of the presently disclosed CaRGOS innovation. Since this can be a significant roadblock to real-world clinical settings, stability studies were performed on a complex matrix-artificial saliva-with a mixture of two enzymatic proteins [i.e., Amylase (pI 6.5) & Lysozyme (pI 10.7)]. Total protein analysis within artificial saliva in the presence of CaRGOS over a period of 2 months in a concentration range of 2.0-2.5 mg/ml was observed. Surprisingly, total protein concentration was similar to conventional-80° C. storage conditions, respectively. Herein, the superior protein-storage at high concentrations (just an order of concentration away, in contrast to 6 orders in previous study) demonstrated within CaRGOS formulations is a paradigm shift in development of a room-temperature based “pre-analytical” preservative solution. In fact, of even higher significance is that the amylase enzyme activity in CaRGOS stored at room temperature was also similar to conventional “optimal”-80° C. storage over a period of 2 months. However, in the process, enzymatic activity of lysozyme had undergone a significant loss. This leads to the scientific premise that lysozyme with pI (10.7) greater than its near-physiological pH environment (7-8) would electrostatically adsorb on negatively charged silica resulting in more protein-unfolding and significant loss of enzymatic activity. This was further validated with miRNA stability in the presence of RNase A, and no degradation of miRNA in presence of the enzyme was observed (see below).

### Example 4

### Evaluation of miRNA Stability in the Presence of RNase

It was hypothesized that long-term stability of miRNA21 could not emanate from repulsive interaction (only), and it was anticipated that some other factors might play a critical role. A key possibility is that the CaRGOS can interact with RNase (which can arise from compromised sterility) and therefore can prevent the denaturation of miRNA by RNase. Previously, Buijs et al. had reported an electrostatic adsorption induced destabilization of proteins (e.g., RNase A/Lysozyme) on 11 nm sized silica particles (36). Also, the non-covalent interactions between biological entities and silica nanomaterials are well-known to electrostatically destabilize nucleases (e.g., RNase) and protease activity as well as providing stability to biospecimens (e.g., lipids, proteins, nucleic acids) in their immobilization matrices (Vertegel et al., 2004; Kandimalla et al., 2006; Shang et al., 2007; Lee et al., 2012; Schlipf et al., 2013; Xiaolin et al., 2015).

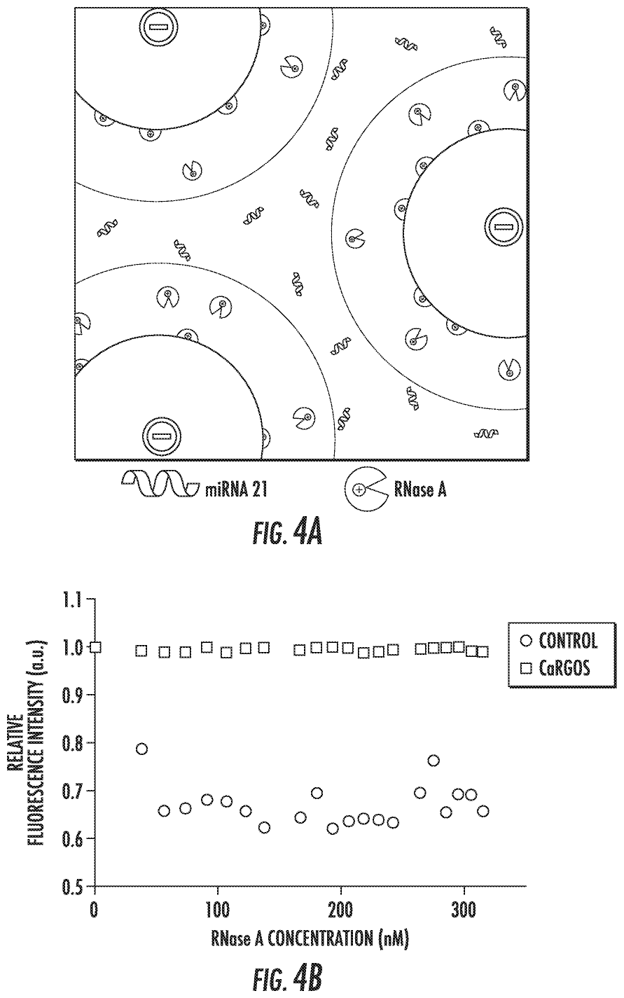

At pH 7.4, RNase A has a asymmetrically stronger positive charge density across the longest axis of the molecule (PDB 2AAS; Lee & Belfort, 1989; Larsericsdotter et al., 2001; Shang et al., 2007). Also, RNase A's active site has been reported to reside in this electropositive potential region. Therefore, and without being bound by any particular theory of operation, the highest miRNA21 expression levels ˜(309-579) nM observed in lower CaRGOS concentrations [(0.5-1.7)% v/v; pH>7] was tentatively attributed to the substantial electrostatic interactions between the positive domain of RNase (sterile-compromised contaminated environment) and negatively charged CaRGOS as shown in FIG. 4A.

These results clearly established that biospecimens can be denatured using CaRGOS if the isoelectric point (pI) of the specimen is higher than that of the pH of the CaRGOS slected, as it will bind to CaRGOS. Similarly, the biospecimen can be stabilized over the long-term if the pI of the specimen is lower than that of the pH of the CaRGOS as it will promote electrostatic repulsion. In fact, most cancer biomarkers, plasma proteins, and immunoglobulins can be preserved using CaRGOS as their pI is lower than 7, thus indicating the wide applicability of CaRGOS in clinical samples (Audain et al., 2016). Simultaneously, the nucleases and proteases that always arise from contamination will be denatured or adsorbed onto the silica particles, thus ensuring the stability of the specific biospecimen. (FIG. 4A) Therefore, the presently disclosed subject matter can be employed for preservation of nucleotides including DNA and RNA as well as most proteins.

To validate this hypothesis, the capability of CaRGOS to prevent degradation of yeast RNA in the presence of bovine pancreatic RNase A was tested (Tripathy et al., 2013). As mentioned herein above and shown in FIG. 4B, the fluorescence emission intensities of yeast RNA-intercalated ethidium bromide [EtBr: 600 nm emission; 510 nm excitation)] solutions in CaRGOS and control buffers were observed in an incrementally increasing RNase A concentration in (0-1200) nM range. Normalized with EtBr's fluorescence emission at 0 nM RNase A concentration, a ˜40% relative fluorescence quenching was observed in control buffer solutions (i.e., without CaRGOS), indicating degradation of yeast RNA with incremental increase in RNase A concentrations (Tripathy et al., 2013). However, unaltered fluorescence emission intensity was observed in CaRGOS buffer solutions within (0-320) nM range of RNase A concentrations (see FIG. 4B). Such unaltered fluorescence emissions were attributed to the electrostatic adsorption inducted RNase A inhibition with CaRGOS (Lee & Belfort, 1989; Santoro et al., 1993; Larsericsdotter et al., 2001; Vertegel et al., 2004; Roach et al., 2006; Shang et al., 2007). In contrast to low RNase A concentration range (0-320) nM, an increase in relative fluorescence emission intensities was observed in (320-1200) nM RNase A concentrations range. This re-increase in fluorescence emission of ethidium bromide in high RNase concentration range was attributed to EtBr's intercalation with: (i) the CaRGOS sol-gel network imparting restricting mobility; (ii) the remaining non-degraded yeast RNA; and (iii) the large excess of RNase A, respectively (Tripathy et al., 2013).

CaRGOS demonstrated larger hydrodynamic size of ˜69 nm and displayed high stability in their buffer dispersions with zeta potential of ˜−21 mV. Therefore, CaRGOS are an excellent candidate for preventing RNA degradation against RNase resulting from environmental contamination (e.g., bacteria, fungi) during transportation/storage and downstream processing (Mutter et al., 2004; Fabre et al., 2013). Based on this premise, Table 1 shows the pI values of proteins present in plasma along with proteins that can denature biospecimens (e.g., nucleases and proteases). The values shown are evidence for CaRGOS providing a high level of stability as most proteins in plasma and biospecimens including miRNA and hemoglobin have pI's compatible with the CaRGOS formulations. As such, the presently disclosed subject matter provides improved compositions and methods for RNA storage at room/elevated temperatures by demonstrating: (a) RNase inhibition; and (b) restriction of miRNA backbone mobility in CaRGOS, respectively.

TABLE 1
pI Values of Proteins Present in Plasma Along with Proteins
that Can Denature Biospecimens (Nucleases and Proteases)

| Protein | pI |
|---|---|
| Albumin | 4.88 |
| A-globulin | 5.60 |
| B-globulin | 5.12 |
| γ-globulin | 6.7 |
| Fibrogen | 5.8 |
| Lysozyme | 11.0 |

### Example 5

### Polyethylene Glycol-Induced Hemoglobin Release in CaRGOS Formulations

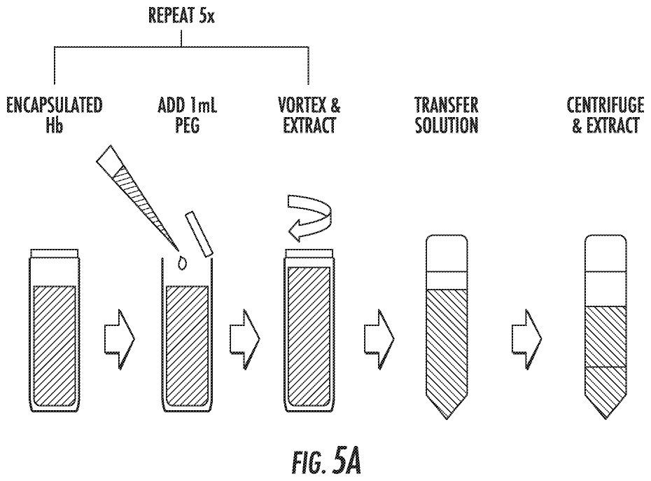

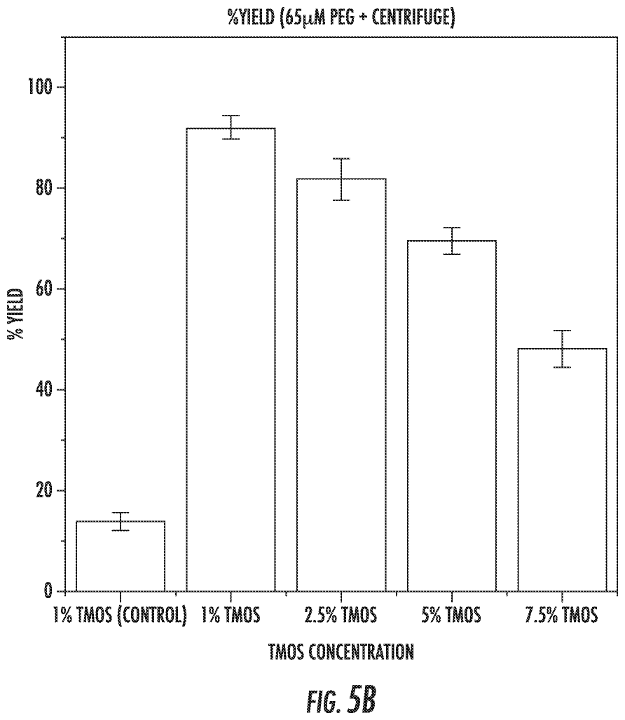

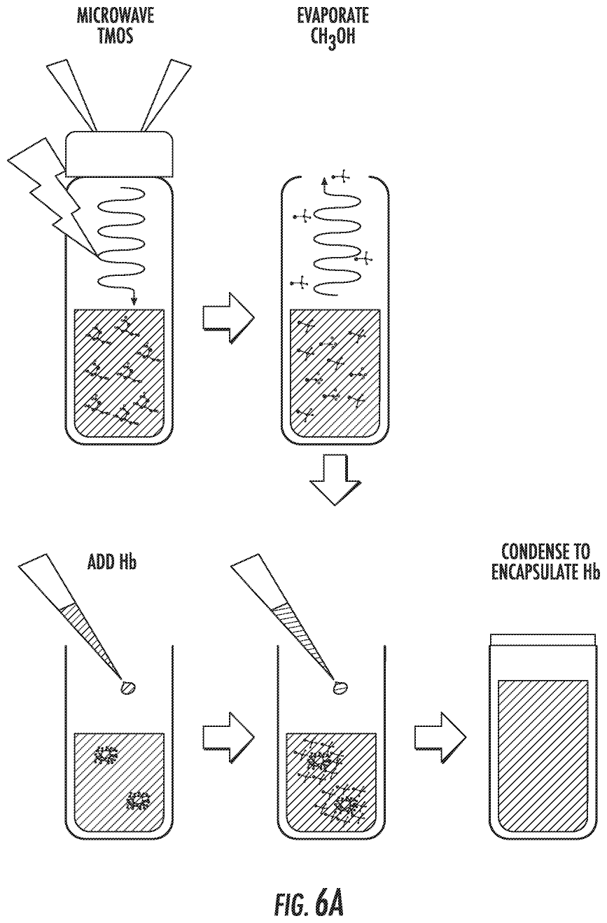

The development of a biocompatible release protocol is desirable as it allows users to run diagnostics on the extracted proteins. Post-encapsulation of hemoglobin within CaRGOS matrices, PEG was systematically added to all CaRGOS formulations as shown in FIG. 5A. A quick re-dissolution of low-to-high viscous CaRGOS-hemoglobin formulations [(1.0-7.5)% v/v TMOS; 0.01 wt./v % Hemoglobin; 0.5 M PB, pH 8.2; 3.0 mL] was observed upon addition of polyethylene glycol [PEG (65 μM, 2 kDa)]. Upon centrifuging the dissolved CaRGOS samples and extracting the supinated solution, a three- to five-fold increase in hemoglobin's UV-Vis absorbance (406 nm) was observed in the resulting solution (FIG. 5B). This large increment in the absorbance intensity of heme group [406 nm] was attributed to a synergistic hydrophilicity imparted by PEG (260 nm) to the CaRGOS formulations, indicating a facile passage and release of hemoglobin throughout the CaRGOS matrices without any loss of protein nativity as shown in FIGS. 5A and 5B. Particularly, an ideal ensilication matrix allowed efficient bioanalyte immobilization (i.e., encapsulation entrapment and/or collaterally depositing) and a facile passage without any physical rupture. Therefore, the highly porous and moderately viscous CaRGOS formulations disclosed herein met these standards, due at least in part to the long-term storage capabilities of CaRGOS and the biocompatible PEG release protocol as shown in FIG. 5A.

### Discussion of the EXAMPLES

Storage of biospecimens in their near native environment at room temperature can have a transformative global impact, however, to do so remains an arduous challenge to date due to the rapid degradation of biospecimen over time. Currently, most isolated biospecimens are refrigerated for short-term storage and frozen (−20° C., −80° C., liquid nitrogen) for long-term storage. An aqueous storage solution that can preserve the biospecimen nearly “as is” had not yet been described.

Disclosed herein are aqueous Capture and Release Gels for optimized storage (Bio-CaRGOS) of biospecimens. Complete recovery of the highly sensitive cancer biomarker miRNA21 at 4° C., 25° C., and 40° C. over a period of ˜3 months and 95% recovery of hemoglobin at 25° C. (1-month) and 96% recovery (7-months) at 4° C. have been demonstrated. In contrast, the control miRNA samples completely degraded in less than 1 week and two-thirds of the control hemoglobin samples degraded in less than one month at 25° C. and seven months at 4° C.). The presently disclosed subject matter is facile, reproducible, and can achieve stabilization of any biospecimen of interest, including but not limited to RNA, DNA, and proteins within just a few minutes using a standard benchtop microwave.

### REFERENCES

All references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Audain et al. (2016) Accurate estimation of isoelectric point of protein and peptide based on amino acid sequences. Bioinformatics 32:821-827.
Bursell & King (2000) The Potential Use of Glutathionyl Hemoglobin as a Clinical Marker of Oxidative Stress. Clinical Chemistry 46:145-146.
Carrasquilla et al. (2012) Stabilizing Structure-Switching Signaling RNA Aptamers by Entrapment in Sol-Gel Derived Materials for Solid-Phase Assays. Journal of the American Chemical Society 134:10998-11005 (2012).
Cha et al. (2018) Extra-Large Pore Mesoporous Silica Nanoparticles Enabling Co-Delivery of High Amounts of Protein Antigen and Toll-like Receptor 9 Agonist for Enhanced Cancer Vaccine Efficacy. ACS Central Science 4:484-492.
Chen et al. (2017) Thermal stability, storage and release of proteins with tailored fit in silica. Scientific Reports 7:46568.
Chen et al. (2018) Immunomodulatory effects of mesoporous silica nanoparticles on osteogenesis: From nanoimmunotoxicity to nanoimmunotherapy. Applied Materials Today 10:184-193.
Engel et al. (2014) National Cancer Institute Biospecimen Evidence-Based Practices: a novel approach to pre-analytical standardization. Biopreservation and Biobanking 12:148-150.
Fabre et al. (2013) An efficient method for long-term room temperature storage of RNA. European Journal Of Human Genetics 22:379.
Goodarzi et al. (2014) Hemoglobin fructation promotes heme degradation through the generation of endogenous reactive oxygen species. Spectrochimica Acta Part A: Molecular and Biomolecular Spectroscopy 130:561-567.
Harrill et al. (2016) MicroRNA Biomarkers of Toxicity in Biological Matrices. Toxicological Sciences 152:264-272.
Ibberson et al. (2009) RNA degradation compromises the reliability of microRNA expression profiling. BMC Biotechnology 9:102.
Kandimalla et al. (2006) Immobilization of Biomolecules in Sol-Gels: Biological and Analytical Applications. Critical Reviews in Analytical Chemistry 36:73-106.
Kansagara et al. (2008) Dry-state, room-temperature storage of DNA and RNA. Nature Methods 5:iv-v.
Larsericsdotter et al. (2001) Thermodynamic Analysis of Proteins Adsorbed on Silica Particles: Electrostatic Effects. Journal of Colloid and Interface Science 237:98-103.
Lee & Belfort (1989) Changing activity of ribonuclease A during adsorption: a molecular explanation. Proceedings of the National Academy of Sciences of the United States of America 86:8392-8396.
Lee et al. (2012) 3D Porous Sol-Gel Matrix Incorporated Microdevice for Effective Large Volume Cell Sample Pretreatment. Analytical Chemistry 84:4928-4934.
Liu et al. (2015) Evaluation of DNA/RNAshells for Room Temperature Nucleic Acids Storage. Biopreservation and Biobanking 13:49-55.
Lou et al. (2014) A review of room temperature storage of biospecimen tissue and nucleic acids for anatomic pathology laboratories and biorepositories. Clinical Biochemistry 47:267-273.
Martinez et al. (2010) Ambient temperature storage of RNA in GenTegra™ for use in RT-qPCR. BioTechniques 48:328-329.
Meng et al. (2010) Engineered Design of Mesoporous Silica Nanoparticles to Deliver Doxorubicin and Pgp siRNA to Overcome Drug Resistance in a Cancer Cell Line. ACS Nano 4:4539-4550.
Mutter et al. (2004) Comparison of frozen and RNALater solid tissue storage methods for use in RNA expression microarrays. BMC Genomics 5:88.
Peng et al. (2018) Metal-organic frameworks for precise inclusion of single-stranded DNA and transfection in immune cells. Nature Communications 9:1293.
Perumal et al. (2014) Sol-gel processed mupirocin silica microspheres loaded collagen scaffold: A synergistic bio-composite for wound healing. European Journal of Pharmaceutical Sciences 52:26-33.
Pinto et al. (2014) Human plasma stability during handling and storage: impact on NMR metabolomics. Analyst 139:1168-1177.
Raynal et al. (2014) Quality assessment and optimization of purified protein samples: why and how? Microbial Cell Factories 13:180.
Roach et al. (2006) Surface Tailoring for Controlled Protein Adsorption: Effect of Topography at the Nanometer Scale and Chemistry. Journal of the American Chemical Society 128:3939-3945.
Santoro et al. (1993) High-resolution Three-dimensional Structure of Ribonuclease A in Solution by Nuclear Magnetic Resonance Spectroscopy. Journal of Molecular Biology 229:722-734.
Schlipf et al. (2013) Pore-Size Dependent Protein Adsorption and Protection from Proteolytic Hydrolysis in Tailored Mesoporous Silica Particles. ACS Applied Materials & Interfaces 5:10111-10117.
Shang et al. (2007) Unfolding of Ribonuclease A on Silica Nanoparticle Surfaces. Nano Letters 7:1991-1995.
Shaw et al. (2001) The effect of net charge on the solubility, activity, and stability of ribonuclease Sa. Protein Sci 10:1206-1215.
Shen et al. (2011) Mesoporous silica nanoparticles loading doxorubicin reverse multidrug resistance: performance and mechanism. Nanoscale 3:4314-4322.
Silsirivanit (2019) Chapter Five: Glycosylation markers in cancer. In Advances in Clinical Chemistry, Makowski (ed). Elsevier, New York, N.Y., United States of America 89:189-213.
Stevenson et al. (2015) Long-term Stability of Total RNA in RNAstable(®) as Evaluated by Expression Microarray. Biopreservation and Biobanking 13:114-122.
Tripathy et al. (2013) A simple assay for the ribonuclease activity of ribonucleases in the presence of ethidium bromide. Analytical Biochemistry 437:126-129.
Vertegel et al. (2004) Silica Nanoparticle Size Influences the Structure and Enzymatic Activity of Adsorbed Lysozyme. Langmuir 20:6800-6807.
Wagner-Golbs et al. (2019) Effects of Long-Term Storage at-80° C. on the Human Plasma Metabolome. Metabolites 9:99-113.
Xiaolin et al. (2015) Sol-gel Encapsulation of Biomolecules and Cells for Medicinal Applications. Current Topics in Medicinal Chemistry 15:223-244.
Zerda & Hoang (1989) High-Pressure Raman-Study of the hydrolysis reaction in tetramethylorthosilicate (TMOS) Journal of Non-Crystalline Solids 109:9-17.
Zhu et al. (2002) Structural electrochemical study of hemoglobin by in situ circular dichroism thin layer spectroelectrochemistry. Biophysical Chemistry 97:129-138.
Zhu et al. (2014) MiR-20b,-21, and-130b inhibit PTEN expression resulting in B7-H1 over-expression in advanced colorectal cancer. Human Immunology 75:348-353.

It will be understood that various details of the presently disclosed subject matter can be changed without departing from the scope of the presently disclosed subject matter. Furthermore, the foregoing description is for the purpose of illustration only, and not for the purpose of limitation.

1. A method for producing a Capture and Release Gel (CaRGOS) composition, the method comprising:
(a) providing a solution of about 0.5 to about 20% (v/v) tetramethoxy silane (TMOS) and/or a derivative thereof, optionally wherein the solution is an aqueous solution of 0.5 to about 20% (v/v) tetramethoxy silane (TMOS) and/or a derivative thereof in water, optionally nuclease-free and/or protease-free water, or is a low salt aqueous solution, further optionally wherein the TMOS and/or the derivative thereof is at a concentration of about 0.5-10.0% (v/v);
(b) heating the solution for a time and at a temperature sufficient to solubilize and at least partially hydrolyze the TMOS and/or the derivative thereof in the solution, impart sterility to the solution, and/or evaporate all or substantially all methanol present and/or generated in the solution; and
(c) adding a buffer to the heated and at least partially hydrolyzed TMOS and/or derivative thereof, wherein the buffer comprises about 0.01-600 mM salt and/or has a pH of from about 5.0-9.0, and optionally further comprises 1-10 mM EDTA, to produce a buffered TMOS and/or derivative thereof solution,
wherein a Capture and Release Gel (CaRGOS) composition is produced.

2. The method of claim 1, wherein the heating step:
(a) is performed in a microwave oven, optionally for about 15-120 seconds; and/or
(b) raises the temperature of the solution to at least about 40° C., at least about 42° C., at least about 45° C., at least about 50° C., at least about 55° C., at least about 60° C., at least about 64.5° C., at least about 70° C., at least about 75° C., at least about 80° C., at least about 85° C., at least about 90° C., at least about 95° C., or at least about 100° C.

3. The method of claim 1, wherein the CaRGOS composition further comprises a biospecimen.

4. The method of claim 1, wherein the biospecimen is selected from the group consisting of a nucleic acid, optionally an RNA, further optionally a miRNA; a protein, optionally an antibody or a fragment or derivative thereof; a peptide, optionally a peptide hormone; a small molecule, optionally a small molecule drug; a liposome, optionally a liposome encapsulating an active agent; a forensic sample; and a cell and/or a lysate and/or a fraction thereof, or any combination thereof.

5. The method of claim 1, wherein the pH of the CaRGOS composition is about 7.0-8.0, optionally about 7.4-7.6.

6. A CaRGOS composition produced by the method of claim 1.

7. A method for stabilizing a biospecimen to degradation, optionally to nuclease and/or protease degradation, the method comprising:
(a) providing a buffered tetramethoxy silane (TMOS) and/or derivative solution, wherein the buffered (TMOS) and/or derivative solution is produced by:
(i) providing a solution of about 0.5 to about 20% (v/v) tetramethoxy silane (TMOS) and/or a derivative thereof, optionally wherein the solution is an aqueous solution of 0.5 to about 20% (v/v) tetramethoxy silane (TMOS) and/or a derivative thereof in water, optionally nuclease-free and/or protease-free water, or is a low salt aqueous solution, further optionally wherein the TMOS and/or the derivative thereof is at a concentration of about 0.5-10.0% (v/v);
(ii) heating the solution for a time and at a temperature sufficient to solubilize and at least partially hydrolyze the TMOS and/or the derivative thereof in the solution, impart sterility to the solution, and/or evaporate all or substantially all methanol present and/or generated in the solution; and
(iii) adding a buffer to the heated and at least partially hydrolyzed TMOS and/or derivative thereof to produce a CaRGO composition, wherein the buffer comprises about 0.01-600 mM salt and/or has a pH of from about 5.0-9.0, and optionally further comprises 1-10 mM EDTA; and
(b) adding a biospecimen to the CaRGO composition, wherein the biospecimen is provided as an aqueous or low salt suspension or solution, whereby the biospecimen is stabilized against degradation.

8. The method of claim 7, wherein the biospecimen is stabilized against nuclease and/or protease degradation.

9. The method of claim 7, wherein the biospecimen is stabilized against degradation at a temperature of from about 4° C. to about 65° C. for at least 48 hours, for at least 1 week, for at least 2, weeks, or for at least 4 weeks.

10. A kit for storing a degradation-sensitive biospecimen, the kit comprising:
(a) a first container comprising a solution of about 0.5 to about 20% (v/v) tetramethoxy silane (TMOS) and/or a derivative thereof, optionally wherein the solution is an aqueous solution of 0.5 to about 20% (v/v) tetramethoxy silane (TMOS) and/or a derivative thereof in water, optionally nuclease-free and/or protease-free water, or is a low salt aqueous solution, further optionally wherein the TMOS and/or the derivative thereof is at a concentration of about 0.5-10.0% (v/v); and optionally one or more of:
(i) a low salt buffer comprising 0.05-0.6 M NaCl; and/or
(ii) 1-1000 mM Tris-HCl (pH 5.0-9.0); and/or
(iii) 1-10 mM EDTA ; and/or
(iv) nuclease-free and/or protease-free water, wherein the low salt buffer and the nuclease-free and/or protease-free water are present in separate containers; and
(b) instructions for using the contents of the kit for storing a nuclease-sensitive and/or protease-sensitive biospecimen.

11. A composition for storing a biospecimen, the composition comprising:
(a) 0.5-20% (v/v) silicic acid;
(b) 0.05-0.6 M salt; and
(c) a buffer that maintains the composition at a pH of about 5.0-9.0.

12. The composition of claim 11, wherein the composition further comprises a biospecimen.

13. The composition of claim 12, wherein the biospecimen is selected from the group consisting of a nucleic acid, optionally an RNA, further optionally a miRNA; a protein, optionally an antibody or a fragment or derivative thereof; a peptide, optionally a peptide hormone; a small molecule, optionally a small molecule drug; a liposome, optionally a liposome encapsulating an active agent; a forensic sample; and a cell and/or a lysate and/or a fraction thereof, or any combination thereof.

14. The composition of claim 12, wherein the biospecimen is a nucleic acid, and the silicic acid is present in the composition at a concentration of about 0.05-10% (v/v).

15. The composition of claim 12, wherein the biospecimen is a peptide or polypeptide, and the silicic acid is present in the composition at a concentration of about 5.0-20% (v/v).

16. The composition of claim 15, wherein the pH of the composition is lower than the pl of the peptide or polypeptide.
